(19) United States
(12) Patent Application Publication
(10) Pub. No.: US 2025/0313523 A1
(43) Pub. Date: Oct. 9, 2025

(54) FRAGRANCE AND FLAVOR COMPOSITIONS COMPRISING NORBORNANE AND NORBORNENE DERIVATIVES

(71) Applicant: OSMO LABS, PBC, New York, NY (US)

(72) Inventors: Andrew P. PATRON, Oceanside, CA (US); Gregory L. WARREN, Winchester, MA (US); Bradley B. GILBERT, Milford, NJ (US); Benjamin AMORELLI, Brielle, NJ (US)

(21) Appl. No.: 19/169,919

(22) Filed: Apr. 3, 2025

Related U.S. Application Data
(60) Provisional application No. 63/574,244, filed on Apr. 3, 2024.

(51) Int. Cl. C07C 69/013; A23L 27/20; C07C 35/20; C07C 43/184; C07D 307/79; C07D 311/20; C11B 9/00

(52) U.S. Cl. CPC C07C 69/013; A23L 27/2026; A23L 27/2028; C07C 35/20; C07C 43/184; C07D 307/79; C07D 311/20; C11B 9/0042; C11B 9/0076; C11B 9/008

(57) ABSTRACT

The present application relates to bicyclo[2.2.1]hept-5-en-2-ol and related compounds, methods of making them, and methods of using them as flavor and fragrance ingredients in food, cosmetic, pharmaceutical, consumer, and other compositions and products.

### CROSS-REFERENCE TO RELATED APPLICATIONS

This application is an U.S. application filed under 35 U.S.C. § 111(a) which claims priority to, and the benefit of, U.S. Provisional Applic. Ser. No. 63/574,244, filed on Apr. 3, 2024, the contents of which are hereby incorporated by reference in its entirety.

### FIELD OF THE APPLICATION

The present application relates to bicyclo[2.2.1]hept-5-en-2-ol and related compounds, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Toasted coconut and other nutty smells may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

Using a proprietary predictive method, the inventors have identified 2 bicyclo[2.2.1]hept-5-en-2-ol, and related compounds, as new ingredients useful in flavors and fragrances. Such compounds have not been specifically identified as a flavor or fragrance ingredient, nor have their smells or odors been previously described.

This application describes the surprising and unexpected olfactive qualities of norbornane and norbornene derivatives, such as bicyclo[2.2.1]hept-5-en-2-ol, and analogs and derivatives thereof, and their use as fragrance and flavor ingredients, and potential applications thereof.

### BRIEF SUMMARY

In one aspect, the application relates to compounds of Formula I or compounds of Formula II:

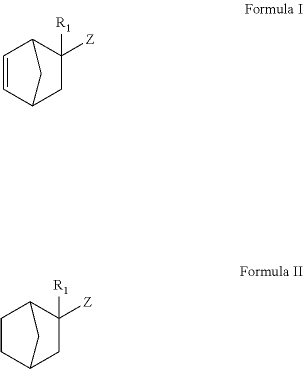

wherein:
R¹ is H or C₁₋₆alkyl, C₂₋₆alkenyl, or C₃₋₆cycloalkyl; or R¹ and Z together form a lactone ring, wherein said lactone ring is optionally substituted by C₁₋₃alkyl and optionally unsaturated;
Z is selected from OR, O—C₂₋₆alkylene-OR, OC(═O)R, O—C₁₋₆alkylene-C(═O)R, O—C₁₋₆alkylene-C(═O)OR; and
R is H, aryl, C₁₋₆alkyl, C₂₋₆alkenyl, or C₃₋₆cycloalkyl;
optionally provided that the compound is not bicyclo[2.2.1]hept-5-en-2-ol.

In some embodiments, the compound of Formula I is bicyclo[2.2.1]hept-5-en-2-ol.

In another aspect, the application relates to fragrance and flavor compositions comprising the compound of Formula I or II, optionally comprising one or more additives, additional fragrance or flavor ingredients, or a combination of additives and fragrance or flavor ingredients. In some embodiments, the application relates to fragrance and flavor compositions comprising bicyclo[2.2.1]hept-5-en-2-ol.

In another aspect, the application relates to products, such as consumer products, comprising such fragrance and flavor compositions comprising the compound of Formula I or II as herein provided, e.g., products comprising bicyclo[2.2.1]hept-5-en-2-ol.

In another aspect, the present disclosure provides a method of making compounds of Formula I or II.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that the compound bicyclo[2.2.1]hept-5-en-2-ol has a unique and favorable aroma. It has thus been determined to be useful in imparting and providing desirable aromas and/or flavors to the products to which it is added. Other Compounds of Formula I or Il are expected to likewise have pleasant or desirable flavors and/or aromas. Such Compounds are therefore potentially useful for products where the inclusion of a pleasing fragrance or flavor is desired, including, but not limited to, perfumes, household products, laundry products, personal care products, cosmetics, dental hygiene products, orally administered medications, and food products. The Compounds of Formula I or II may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula I or Formula II:

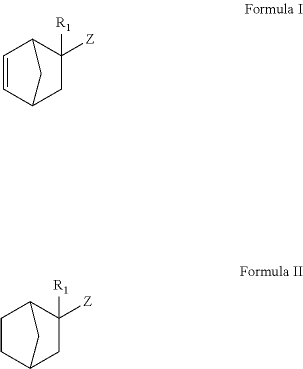

wherein:
R¹ is H or C₁₋₆alkyl, C₂₋₆alkenyl, or C₃₋₆cycloalkyl; or R¹ and Z together form a lactone ring, wherein said lactone ring is optionally substituted by C₁₋₃alkyl and optionally unsaturated;
Z is selected from OR, O—C₂₋₆alkylene-OR, OC(═O)R, O—C₁₋₆alkylene-C(═O)R, O—C₁₋₆alkylene-C(═O)OR; and
R is H, aryl, C₁₋₆alkyl, C₂₋₆alkenyl, or C₃₋₆cycloalkyl;
optionally provided that the compound is not bicyclo[2.2.1]hept-5-en-2-ol.

In further embodiments, of the first aspect, the present disclosure provides:
1.1 Compound of Formula I, wherein R¹ is H;
1.2 Compound of Formula I, wherein R¹ is C₁₋₆alkyl (e.g., methyl, ethyl);
1.3 Compound 1.2, wherein R¹ is methyl;
1.4 Compound of Formula I, wherein R¹ is C₃₋₆cycloalkyl (e.g., cyclopropyl);
1.5 Compound of Formula I, wherein R¹ is C₂₋₆alkenyl (e.g., vinyl, allyl, 2-methylallyl), optionally wherein said C₂₋₆alkenyl has (E) or (Z) stereochemistry;
1.6 Compound 1.5, wherein R¹ is vinyl;
1.7 Compound 1.5, wherein R¹ is allyl;
1.8 Compound of Formula I, wherein R¹ and Z together form a lactone ring, optionally substituted by C₁₋₃alkyl (e.g., methyl);
1.9 Compound 1.8, wherein said lactone ring is a 5- or 6-membered lactone ring;
1.10 Compound 1.8, wherein said lactone ring is a saturated 5-membered lactone ring (e.g., a dihydro-2(3H)-one ring);
1.11 Compound 1.8, wherein said lactone ring is an unsaturated 5-membered lactone ring (e.g., a furan-2(5H)-one ring);
1.12 Compound 1.8, wherein said lactone ring is a saturated 6-membered lactone ring (e.g., a tetrahydro-2H-pyran-2-one ring);
1.13 Compound 1.8, wherein said lactone ring is an unsaturated 6-membered lactone ring (e.g., a 5,6-dihydro-2H-pyran-2-one ring);
1.14 Compound of Formula I, or any of 1.1-1.13, wherein Z is OR;
1.15 Compound of Formula I, or any of 1.1-1.13, wherein Z is O—C₂₋₆alkylene-OR;
1.16 Compound 1.15, wherein the C₂₋₆alkylene moiety is selected from —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.17 Compound of Formula I, or any of 1.1-1.13, wherein Z is OC(═O)R;
1.18 Compound of Formula I, or any of 1.1-1.13, wherein Z is O—C₁₋₆alkylene-C(═O)R;
1.19 Compound 1.18, wherein the C₁₋₆alkylene moiety is selected from —CH₂—, —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.20 Compound of Formula I, or any of 1.1-1.13, wherein Z is O—C₁₋₆alkylene-C(═O)OR;
1.21 Compound 1.20, wherein the C₁₋₆alkylene moiety is selected from —CH₂—, —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.22 Compound of Formula I, or any of 1.1-1.21, wherein R is H;
1.23 Compound of Formula I, or any of 1.1-1.21, wherein R is aryl (e.g., phenyl);
1.24 Compound of Formula I, or any of 1.1-1.21, wherein R is C₁₋₆alkyl (e.g., methyl, ethyl, propyl, isopropyl, sec-butyl, isobutyl, or tert-butyl);
1.25 Compound of Formula I, or any of 1.1-1.21, wherein R is C₂₋₆alkenyl (e.g., vinyl, allyl, crotyl, methallyl, 3,3-dimethylallyl, 1-propen-1-yl, 2-buten-1-yl, 2-buten-2-yl, 2-penten-2-yl, 3-methyl-2-buten-1-yl), optionally wherein each of said C₂₋₆alkenyl has (E) or (Z) stereochemistry;
1.26 Compound of Formula I, or any of 1.1-1.21, wherein R is C₃₋₆cycloalkyl (e.g., cyclopropyl, cyclobutyl);
1.27 Compound of Formula I, or any of 1.1-1.26, wherein Z is OC(═O)R and R is selected from methyl, ethyl, propyl, isopropyl, tert-butyl, 1-propen-1-yl, 2-buten-2-yl, and 2-penten-2-yl (e.g., (E)-1-propen-1-yl, (E)-2-buten-2-yl, or (E)-2-penten-2-yl);
1.28 Compound of Formula I, or any of 1.1-1.26, wherein Z is OR and R is selected from methyl, ethyl, allyl, methallyl, and 3-methyl-2-buten-1-yl (e.g., (E)-3-methyl-2-buten-1-yl);
1.29 Compound of Formula I, or any of 1.1-1.26, wherein Z is OR and R is H;
1.30 Compound of Formula I, or any of 1.1-1.29, wherein the compound of Formula I is selected from any of the following:

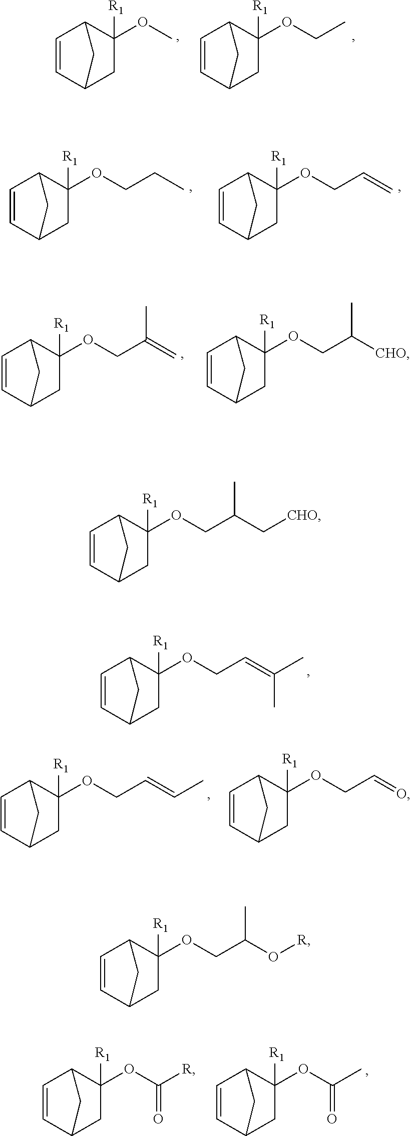

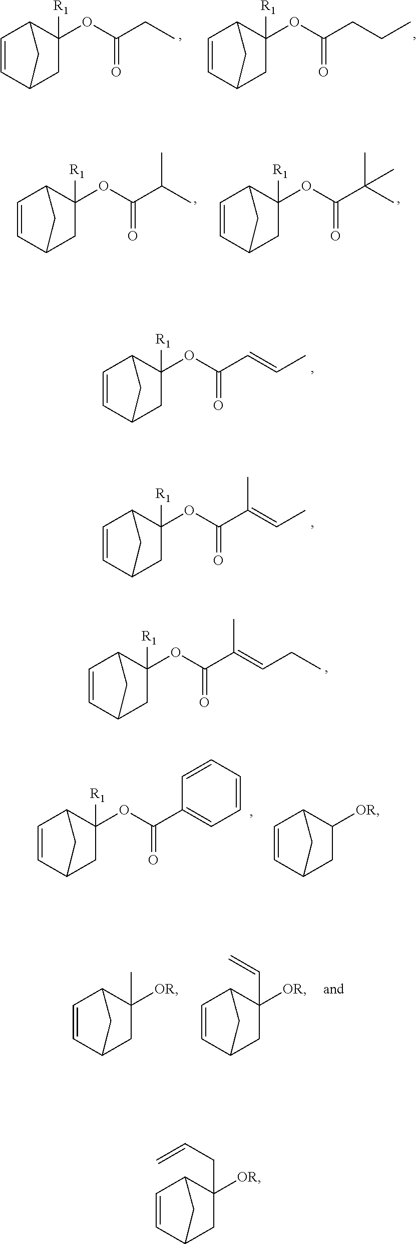

wherein R¹ and R, are as defined in any of the preceding formulae (e.g., wherein R¹ and R, are each independently H, methyl, or ethyl);
1.31 Compound of Formula I, or any of 1.1-1.29, wherein the compound of Formula I is selected from any of the following:

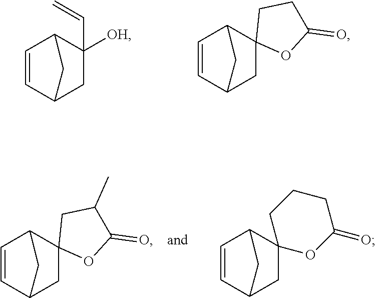

1.32 Compound of Formula I, or any of 1.1-1.29, wherein the compound of Formula I is selected from any of the following:

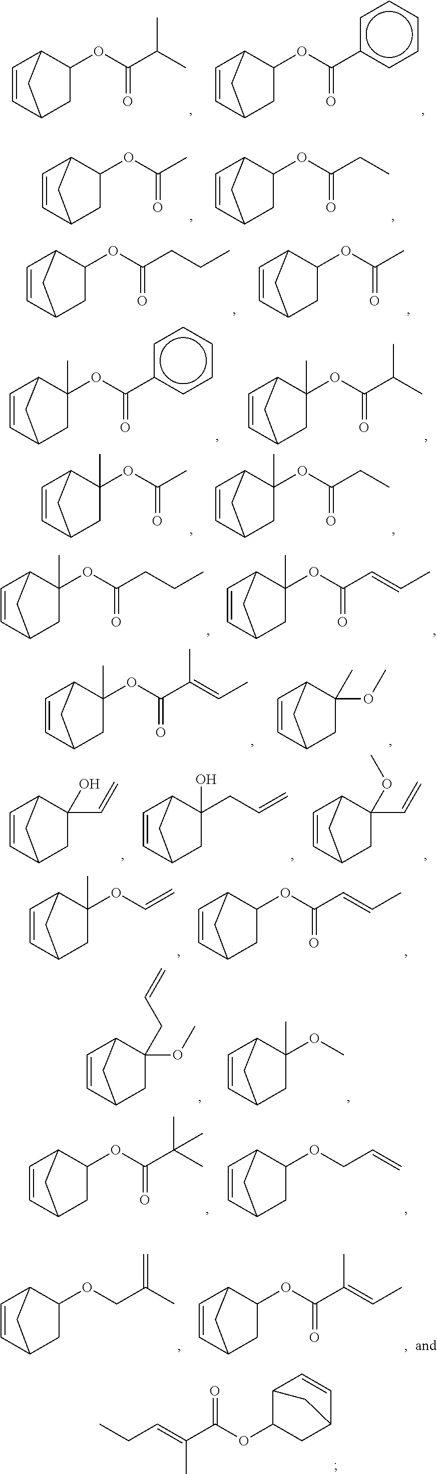

1.33 Compound of Formula I, or any of 1.1-1.29, wherein the compound of Formula I is selected from any of the following:

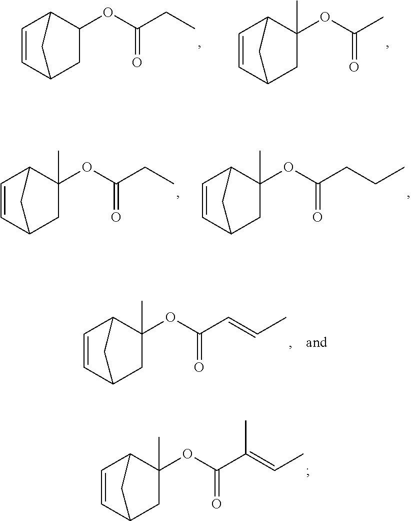

1.34 Compound of Formula I or any of 1.1-1.32, wherein the compound is not 2 bicyclo[2.2.1]hept-5-en-2-ol, optionally wherein the compound is not 5-norbornen-2-yl acetate;
1.35 Compound of Formula I or any of 1.1-1.32, wherein the compound is bicyclo[2.2.1]hept-5-en-2-ol;
1.36 Compound of Formula II, wherein R¹ is H;
1.37 Compound of Formula II, wherein R¹ is C₁₋₆alkyl (e.g., methyl, ethyl);
1.38 Compound of Formula II, wherein R¹ is methyl;
1.39 Compound of Formula II, wherein R¹ is C₃₋₆cycloalkyl (e.g., cyclopropyl);
1.40 Compound of Formula II, wherein R¹ is C₂₋₆alkenyl (e.g., vinyl, allyl, 2-methyallyl), optionally wherein said C₂₋₆alkenyl has (E) or (Z) stereochemistry;
1.41 Compound 1.40, wherein R¹ is vinyl;
1.42 Compound 1.40, wherein R¹ is allyl;
1.43 Compound of Formula II, wherein R¹ and Z together form a lactone ring, optionally substituted by C₁₋₃alkyl (e.g., methyl);
1.44 Compound 1.43, wherein said lactone ring is a 5- or 6-membered lactone ring;
1.45 Compound 1.44, wherein said lactone ring is a saturated 5-membered lactone ring (e.g., a dihydro-2(3H)-one ring);
1.46 Compound 1.44, wherein said lactone ring is an unsaturated 5-membered lactone ring (e.g., a furan-2(5H)-one ring);
1.47 Compound 1.44, wherein said lactone ring is a saturated 6-membered lactone ring (e.g., a tetrahydro-2H-pyran-2-one ring);
1.48 Compound 1.44, wherein said lactone ring is an unsaturated 6-membered lactone ring (e.g., a 5,6-dihydro-2H-pyran-2-one ring);
1.49 Compound of Formula II, or any of 1.36-1.48, wherein Z is OR;
1.50 Compound of Formula II, or any of 1.36-1.48, wherein Z is O—C₂₋₆alkylene-OR;
1.51 Compound 1.50, wherein the C₂₋₆alkylene moiety is selected from —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.52 Compound of Formula II, or any of 1.36-1.48, wherein Z is OC(═O)R;
1.53 Compound of Formula II, or any of 1.36-1.48, wherein Z is O—C₁₋₆alkylene-C(═O)R;
1.54 Compound 1.53, wherein the C₁₋₆alkylene moiety is selected from —CH₂—, —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.55 Compound of Formula II, or any of 1.36-1.48, wherein Z is O—C₁₋₆alkylene-C(═O)OR;
1.56 Compound 1.55, wherein the C₁₋₆alkylene moiety is selected from —CH₂—, —CH₂CH₂—, —CH₂CH₂CH₂—, —CH(CH₃)CH₂—, —CH₂CH(CH₃)—, —CH₂CH₂CH₂CH₂—, —CH₂CH(CH₃)CH₂—, CH(CH₃)CH₂CH₂—, and CH₂CH₂CH(CH₃)—;
1.57 Compound of Formula II, or any of 1.36-1.56, wherein R is H;
1.58 Compound of Formula II, or any of 1.36-1.56, wherein R is aryl (e.g., phenyl);
1.59 Compound of Formula II, or any of 1.36-1.56, wherein R is C₁₋₆alkyl (e.g., methyl, ethyl, propyl, isopropyl, sec-butyl, isobutyl, or tert-butyl);
1.60 Compound of Formula II, or any of 1.36-1.56, wherein R is C₂₋₆alkenyl (e.g., vinyl, allyl, crotyl, methallyl, 3,3-dimethylallyl, 1-propen-1-yl2-buten-1-yl, 2-buten-2-yl, 2-penten-2-yl, 3-methyl-2-buten-1-yl), optionally wherein each of said C₂₋₆alkenyl has (E) or (Z) stereochemistry;
1.61 Compound of Formula II, or any of 1.36-1.60, wherein R is C₃₋₆cycloalkyl (e.g., cyclopropyl, cyclobutyl);
1.62 Compound of Formula II, or any of 1.36-1.61, wherein Z is OC(═O)R and R is selected from methyl, ethyl, propyl, isopropyl, tert-butyl, 1-propen-1-yl, 2-buten-2-yl, and 2-penten-2-yl (e.g., (E)-1-propen-1-yl, (E)-2-buten-2-yl, or (E)-2-penten-2-yl);
1.63 Compound of Formula II, or any of 1.36-1.61, wherein Z is OR and R is selected from methyl, ethyl, allyl, methallyl, and 3-methyl-2-buten-1-yl (e.g., (E)-3-methyl-2-buten-1-yl);
1.64 Compound of Formula II, or any of 1.36-1.61, wherein Z is OR and R is H;
1.65 Compound of Formula II, or any of 1.36-1.64, wherein the compound of Formula II is selected from any of the following:

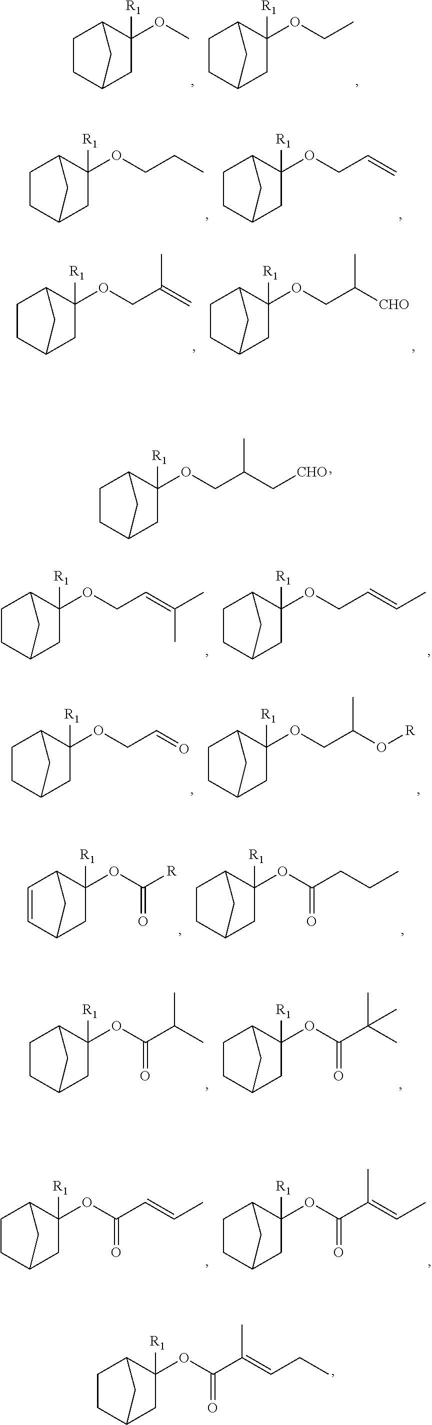

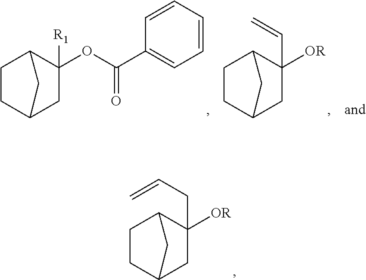

wherein R¹ and R, are as defined in any of the preceding formulae (e.g., wherein R¹ and R, are each independently H, methyl, or ethyl);
1.66 Compound of Formula II, or any of 1.36-1.64, wherein the compound of Formula II is selected from any of the following:

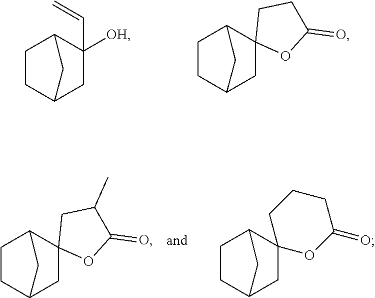

1.67 Compound of Formula II, or any of 1.36-1.64, wherein the compound of Formula II is selected from any of the following:

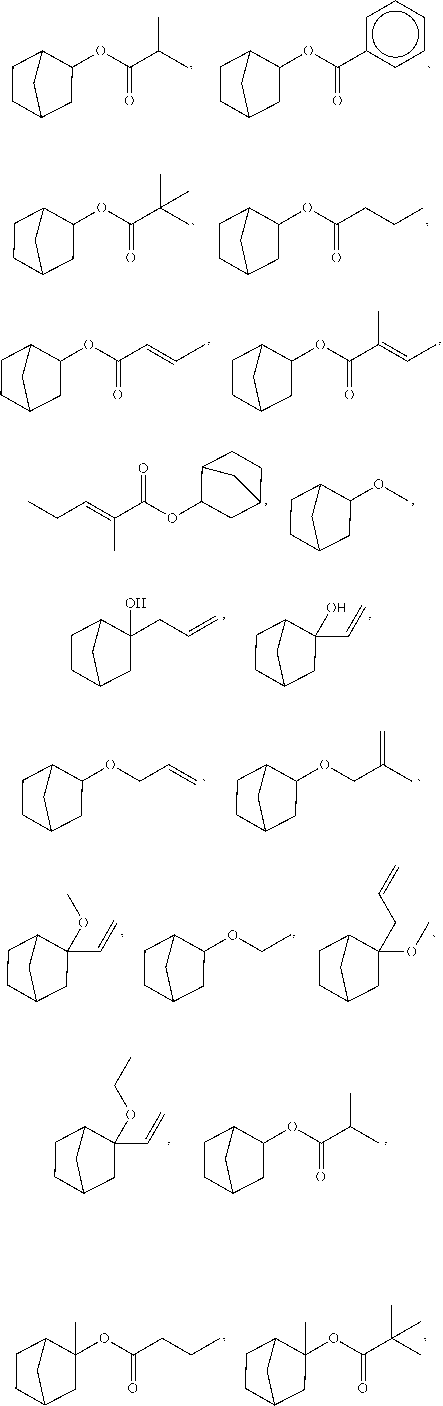

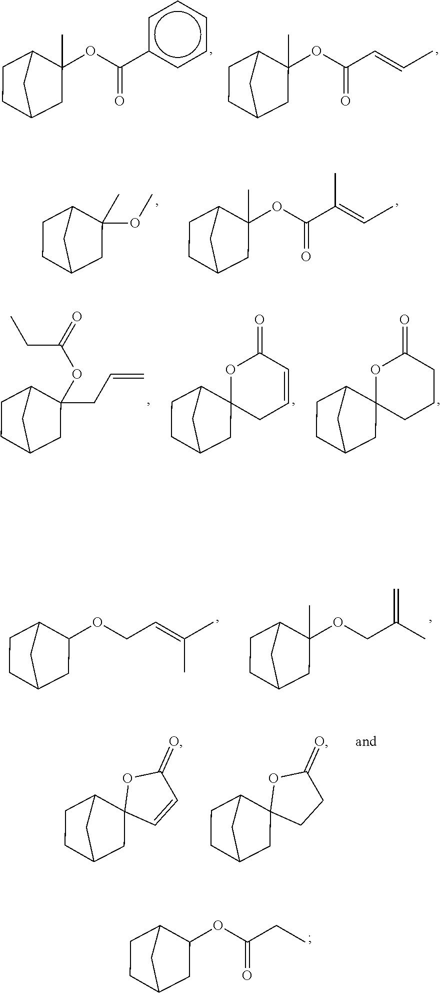

1.68 Compound of Formula II, or any of 1.36-1.64, wherein the compound of Formula II is selected from any of the following:

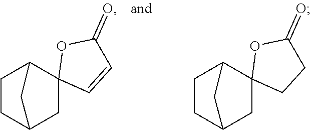 [SMILES: O=C1C=CC2(CC3CCC2C3)O1 ; O=C1CCC2(CC3CCC2C3)O1]

1.69 Any preceding compound, wherein the compound has a molecular weight selected from the range of to 110 to 600, or 110 to 500, or 110 to 450, or 110 to 400, or 110 to 350, or 110 to 300, or 110 to 250, or 110 to 200;
1.70 Any preceding compound, wherein the compound has a pleasing taste and/or aroma, e.g., as judged by a trained flavor or fragrance chemist or master perfumer (e.g., toasted coconut or other nutty aromas and/or tastes).

It is understood that the norbornene core of the Compounds of Formula I have the following standard alternative numberings, depending on the nature of the group R in the following formulas:

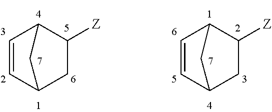

It is further understood that a norbornene core, or any compound comprising same, as drawn and/or described herein, may comprise any one or more of the following stereoisomers:

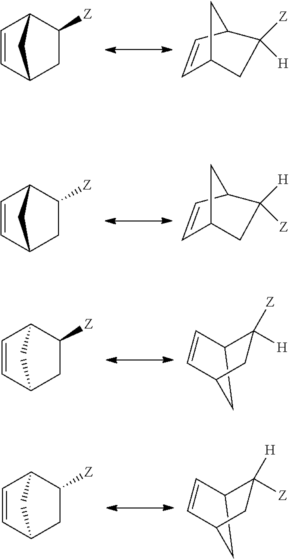

These stereoisomers are distinct from each, as shown below for the stereoisomers of 5-methoxy-2-norbornene:

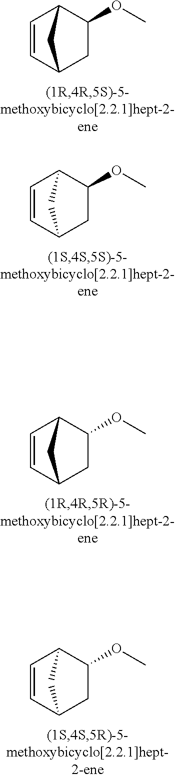

Any compound or structure referenced here may exist in any combination of these stereoisomers, for example as a racemic mixture of enantiomeric pairs, or as equal or non-equal mixtures of any diastereomers, or any other combination.

In a second aspect, the present disclosure provides a flavor composition and/or a fragrance composition (Composition 1) comprising a Compound of Formula I or II, or any of 1.1-1.70, in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound of Formula I or II, or any of 1.1-1.70, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula I or II or any of 1.1-1.70) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredients in the composition and the concentration of the composition used in the product.

As used herein, the term “flavor composition” means a mixture of flavor ingredients (e.g., including a Compound of Formula I or II or any of 1.1-1.70) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formula I or II which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl) furan, 2,3-dihydrofuran, furfural, 5-methylfurfural, 3-(2-furyl)-2-methyl-2-propenal, 5-(hydroxymethyl) furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang ylang, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)-N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl) propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45(2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea, EDTA, polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a fourth aspect, the present disclosure provides a product which comprises Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage ±5%, ±4%, ±3%, ±2.5%, ±2%, ±1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{X}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-pentyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. Exemplary C₁₋₆-alkyl groups include methyl, ethyl, n-propyl, isopropyl, n-butyl, isobutyl, sec-butyl, tert-butyl. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl, allyl, crotyl, methallyl, and 3,3-dimethylallyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

As used herein, the term “heteroaryl” means an aromatic free radical having 5 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### EXAMPLES

Compounds of the present disclosure can be made according to known methods published in the art.

Bicyclo[2.2.1]hept-5-en-2-ol is obtained and its olfactory qualities are studied. Both the neat liquid compound and a 10% dilution of the compound in ethanol are examined by an experienced fragrance chemist (a master perfumer). It is found that the compound exhibits a favorable odor profile characterized by fruity, melon, nice, fleshy, sweet, slightly raw, potato, tomato sauce, tomatillo, overripe melon, and papaya notes.

### Synthetic Examples

The Compounds of Examples 1 to 57 may be prepared according to the procedures described hereinbelow.

### Analytical Method Information

NMR: ¹H-NMR spectra were recorded at 400 MHz on a Bruker Avance AV-I-400 instrument or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

Method 1: UPLC: Waters I-Class; Acq. Method: UPLC_AN_ACID. Column: XSelect CSH C18 XP (50×2.1 mm 2.5μm). Flow: 0.6 ml/min; Column temp: 40° C. Eluent A: 0.1% formic acid in water; Eluent B: 0.1% formic acid in acetonitrile. Gradient: t=0 min 5% B, t=2.0 min 98% B, t=2.7 min 98% B. Post-time: 0.3 min. Detection PDA: 210-320nm and 215nm. Source: ESI; Capillary: 3000 V; Cone: 15 V; Extractor: 3.0 V; RF: 2.5 V; Source Temp.: 150° C.; Desolvation Temp.: 600° C.; Cone Gas Flow: 80 L/Hr; Desolvation Gas Flow: 1000 L/Hr; Full MS scan: MS range 100-800 (positive and negative mode); scan: 0.4 sec.

Method 2: UPLC: Waters I-Class; Acq. Method: UPLC_AN_BASE. Column: XSelect CSH C18 XP (50×2.1 mm 2.5μm). Flow: 0.6 ml/min; Column temp: 25° C. Eluent A: 10 mM ammonium bicarbonate in water (pH 9.5); Eluent B: acetonitrile. Gradient: t=0 min 5% B, t=2 min 98% B, t=2.7 min 98% B. Post-time: 0.3 min. Detection PDA: 210-320nm. Source: ESI; Capillary: 3000 V; Cone: 15 V; Extractor: 3.0 V; RF: 2.5 V; Source Temp.: 150° C.; Desolvation Temp.: 600° C.; Cone Gas Flow: 80 L/Hr; Desolvation Gas Flow: 1000 L/Hr; Full MS scan: MS range 100-800 (positive and negative mode); scan: 0.4 sec.

Method 3: UPLC: Agilent 1290 Infinity II, 1290 G7120A Bin. Pump, 1290 G7167B Multisampler, 1290 MCT G7116B Column Comp., 1290 G7117B PDA (210-320nm), MSD (ESI pos/neg) mass range: 90-1500, G6135B, Column: Atlantis T3(100×3.0 mm, 3μ) Flow: 0.8 ml/min Column temp: 40° C., Eluent A: 0.1% Formic acid in Water, Eluent B: 0.1% Formic acid in Acetonitrile, Gradient: t=0 min 0% B, t=10 min 20% B; t=12 min 20% B2.5 min, Postrun: DAD (210, 336, 210-320nm).

Method 4: UPLC: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5μ) Flow: 0.8 ml/min Column temp: 40° C., Eluent A: 0.1% Formic acid in Water, Eluent B: 0.1% Formic acid in Acetonitrile, Gradient: t=0 min 5% B, t=0.5 min 5% B, t=4.5 min 98% B; t=5 min 98% B, Postrun: 0.5 min. Source: API-ES, Capillary voltage: 3000 V, Drying gas flow: 12 L/min, Nebulizer Pressure 60 psig, Drying Gas Temp: 350° C., Fragmentor 70.

Method 5: UPLC: Waters I-Class UPLC, Binary Solvent Manager (BSM), Sample Manager-FTN (SM-FTN) and Sample Organizer (SO), Column Manager (CM-A), PDA 210-320nm, QDa ESI 100-800 (pos) 100-800 (neg), Column: XSelect CSH C18 XP (50×2.1 mm 2.5μm) Flow: 0.6 ml/min; Column temp: 25° C., Eluent A: 10 mM ammonium bicarbonate in water (pH 9.5), Eluent B: acetonitrile, Gradient: t=0 min 5% B, t=2 min 98% B, t=2.7 min 98% B, Postrun: 0.3 min. Source: ESI; Capillary: 800 V; Cone: 15 V; Source Temp.: 120° C.; Probe Temp.: 600° C.; Full MS scan: MS range 100-800 (positive and negative mode); scan: 0.225 sec.

Method 6: UPLC: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5μ) Flow: 0.8 ml/min Column temp: 25° C., Eluent A: 10 mM Ammonium bicarbonate in Water (pH 9.5), Eluent B: Acetonitrile, Gradient: t=0 min 5% B, t=0.5 min 5% B, t=4.5 min 98% B; t=5 min 98% B, Postrun: 0.5 min. Source: API-ES, Capillary voltage: 3000 V, Drying gas flow: 12 L/min, Nebulizer Pressure 60 psig, Drying Gas Temp: 350° C., Fragmentor 70.

Method 8: GCMS: Instrument: Agilent 8890, G7081B 5977B MSD (EI-positive, Det.temp.: 280° C.) Mass range 50-350, Detection FID/Det.temp: 325° C., Column: Agilent DB-5 MS (20 m ID 180 μm, df 0.18 μm), Average velocity: 39 cm/s, Injection vol: 1 μl, Injector temp: 250° C., Split ratio: 100/1, Carrier gas: He; Initial temp: 40° C., Initial time: 0.5 min, Solvent delay: 1.1 min, Rate 120° C./min, 115° C.; 110° C./min, 175° C.; 80° C./min, Final temp 300° C., Hold time 2.0 min.

Method 9: GCMS: Instrument: Agilent 6890N G1530, G2577A 5973 MSD (EI-positive, Det.temp.: 280° C.) Mass range 50-450, Column: Agilent DB-5 MS (20 m ID 180 μm, df 0.18 μm), Average velocity: 50 cm/s, Injection vol: 1 μl, Injector temp: 250° C., Split ratio: 100/1, Carrier gas: He; Initial temp: 40° C., Initial time: 1.5 min, Solvent delay: 1.8 min, Rate 30° C./min, Final temp 250° C., Hold time 2.0 min.

Method 10: LCMS: Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gasflow: 1.3 ml/min), Column: XSelect CSH C18(30×2.1 mm 3.5μ) Flow: 1 ml/min, Column temp.: 40° C., Eluent A: 0.1% Formic acid in Water, Eluent B: 0.1% Formic acid in Acetonitrile, Gradient: t=0 min 5% B, t=1.6 min 98% B, t=3 min 98% B, Postrun: 1.3 min. Source: API-ES, Capillary voltage: 3000 V, Drying gas flow: 13 L/min, Nebulizer Pressure 60 psig, Drying Gas Temp: 350° C., Fragmentor 70.

Method 11: Instrument: Agilent 6890N G1530, G2577A 5973 MSD (EI-positive, Det.temp.: 280° C.) Mass range 50-550, Column: Agilent DB-5 MS (20 m ID 180 μm, df 0.18 μm), Average velocity: 50 cm/s, Injection vol: 1 μl, Injector temp: 250° C., Split ratio: 100/1, Carrier gas: He; Initial temp: 100° C., Initial time: 1.5 min, Solvent delay: 1.0 min, Rate 75° C./min, Final temp 250° C., Hold time 4.3 min.

### Purification Method Information:

Method 12: Apparatus: Waters Prep 100 SFC UV/MS directed system; Waters 2998 Photodiode Array (PDA) Detector; Waters Acquity QDa MS detector; Waters 2767 Sample Manager; Column: Phenomenex Lux i-Cellulose-5(250×21.2 mm, 5 μm); Column temp: 35° C.; Flow: 70 ml/min; ABPR: 120 bar; Eluent A: CO2, Eluent B: 20 mM Ammonia in methanol; Linear gradient: t=0 min 2% B, t=6 min 10% B, t=7 min 25% B, t=8 min 2% B; Detection: PDA (210-400 nm); Fraction collection: PDA TIC Apparatus: Waters Prep 100 SFC UV/MS directed system; Waters 2998 Photodiode Array (PDA) Detector; Waters Acquity QDa MS detector; Waters 2767 Sample Manager; Column: Phenomenex Lux i-Cellulose-5(250×21.2 mm, 5 μm); Column temp: 35° C.; Flow: 70 ml/min; ABPR: 120 bar; Eluent A: CO2, Eluent B: 20 mM Ammonia in methanol; Linear gradient: t=0 min 2% B, t=6 min 10% B, t=7 min 25% B, t=8 min 2% B; Detection: PDA (210-400 nm); Fraction collection: PDA TIC.

Method 13: Instrument type: Reveleris™ prep MPLC; Column: Dr. Maisch Reprosil C18 150×25 mm, 10μ); Flow: 40 mL/min; Column temp: room temperature; Eluent A: 0.1% (v/v) Formic acid in water, Eluent B: 0.1% (v/v) Formic acid in acetonitrile; Gradient: t=0 min 5% B, t=1 min 5% B, t=2 min 30% B, t=17 min 70% B, t=18 min 100% B, t=23 min 100% B; Detection UV: 220, 254, 340 nm, ELSD.

### Common Intermediates

Intermediate A: Bicyclo[2.2.1]hept-5-en-2-ol

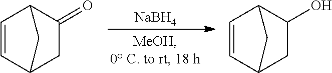 [SMILES: O=C1CC2C=CC1C2 ; "NaBH4" ; OC1CC2C=CC1C2]

To sodium borohydride (6.122 g, 1.75 Eq, 161.8 mmol) in MeOH (200 mL) at 0° C. was added bicyclo[2.2.1]hept-5-en-2-one (10.00 g, 1 Eq, 92.47 mmol) in MeOH (50 mL). After addition, the mixture was allowed to warm to room temperature and stirred for 18h. The mixture was then acidified by addition of 2M aq. HCl and extracted thrice with diethyl ether. The aqueous layer was then saturated by addition of brine and extracted back with diethyl ether. Combined organic layers were washed with brine, dried over Na₂SO₄, filtered and concentrated, affording bicyclo[2.2.1]hept-5-en-2-ol (9.50 g, 86.2 mmol, 93% yield) as an off-white slurry (95:5 mixture of isomers). GCMS: Method 8, 1.90 min, 110.0, calcd. 110.1; ¹H NMR (400 MHz, CDCl₃) δ 6.45 (dd, J=5.9, 3.1 Hz, 1H), 6.07 (dd, J=5.8, 2.9 Hz, 1H), 4.48 (ddd, J=8.1, 3.9, 2.6 Hz, 1H), 3.00 (dq, J=3.6, 1.9 Hz, 1H), 2.82 (dt, J=5.1, 2.5 Hz, 1H), 2.10 (ddd, J=12.1, 8.1, 3.8 Hz, 1H), 1.48 (ddt, J=8.1, 3.9, 2.0 Hz, 1H), 1.31-1.26 (m, 1H), 0.76 (ddd, J=12.5, 3.7, 2.8 Hz, 1H). OH proton not observed.

Intermediate B: 2-Methylbicyclo[2.2.1]hept-5-en-2-ol

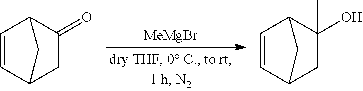

A stirring solution of bicyclo[2.2.1]hept-5-en-2-one (13.0 g, 1 Eq, 0.12 mol) in dry THF (300 mL) was purged with nitrogen and cooled to 0° C. Then methyl magnesium bromide in diethyl ether (0.12 L, 3.0 molar, 3 Eq, 0.36 mol) was added dropwise. After addition, the reaction mixture was allowed to warm to room temperature and stirred for 1 h. The reaction mixture was cooled down on ice and then quenched with aq. sat. NH₄Cl and then concentrated under reduced pressure. The residue was diluted with DCM and a cloudy mixture formed. The mixture was filtered over celite and the filter cake was washed with fresh DCM. Layers were separated and the organics were extracted thrice with additional DCM. The combined organic layers were dried over Na₂SO₄ and concentrated to afford 2-methylbicyclo[2.2.1]hept-5-en-2-ol (13.8 g, 0.11 mol, 92% yield) as a colorless oil (95:5 mixture of isomers). GCMS: Method 8, 1.91 min, 124.0, calcd. 124.1; ¹H NMR (400 MHz, CDCl₃) δ 6.43 (dd, J=5.8, 3.0 Hz, 1H), 6.20 (dd, J=5.8, 3.1 Hz, 1H), 2.83 (d, J=4.3 Hz, 1H), 2.67 (dp, J=2.5, 1.3 Hz, 1H), 1.81 (dd, J=12.3, 3.7 Hz, 1H), 1.57 (d, J=6.9 Hz, 2H), 1.51 (s, 4H), 1.16 (dd, J=12.4, 3.5 Hz, 1H).

Intermediate C: Bicyclo[2.2.1]heptan-2-ol

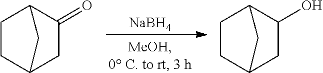 [SMILES: O=C1CC2CCC1C2 ; "NaBH4" ; OC1CC2CCC1C2]

To sodium borohydride (14.02 g, 2.05 Eq, 370.7 mmol) in MeOH (450 mL) at 0° C. was added bicyclo[2.2.1]heptan-2-one (20.00 g, 1 Eq, 181.6 mmol) in MeOH (100 mL). After addition, the mixture was stirred at room temperature for 3h. The mixture was then acidified by addition of 2M aq. HCl and concentrated under reduced pressure. The residue was diluted with diethyl ether and the aqueous layer was extracted with thrice diethyl ether. Combined organics were dried over Na₂SO₄, filtered and evaporated under reduced pressure. The crude was purified by flash column chromatography (SiO₂, 0-50% MTBE in heptane), affording bicyclo[2.2.1]heptan-2-ol (14 g, 125 mmol, 68% yield) as a white solid (95:5 mixture of isomers). GCMS: Method 8, 1.97 min, 112.0, calcd. 112.2; ¹H NMR (400 MHz, CDCl₃) δ 4.23 (d, J=10.0 Hz, 1H), 2.25 (td, J=4.2, 2.1 Hz, 1H), 2.17 (t, J=4.7 Hz, 1H), 1.95 (dddd, J=16.1, 10.8, 5.4, 2.9 Hz, 1H), 1.89-1.83 (m, 1H), 1.55 (ddt, J=11.8, 4.3, 3.0 Hz, 1H), 1.43 (s, 1H), 1.41-1.27 (m, 4H), 0.84 (dt, J=12.9, 3.5 Hz, 1H).

Intermediate D: 2-Methylbicyclo[2.2.1]heptan-2-ol

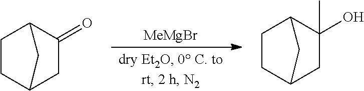

A stirring solution of bicyclo[2.2.1]heptan-2-one (15.00 g, 1 Eq, 136.2 mmol) in dry diethyl ether (400 mL) was purged with nitrogen and cooled down to 0° C. Methyl magnesium bromide solution in dry THF (91 mL, 3.0 molar, 2 Eq, 272 mmol) was added dropwise and after addition, the mixture was allowed to warm to room temperature and stirred for 2h. The reaction mixture was cooled on ice and quenched by addition of aq. sat. NH4Cl and then diluted with diethyl ether. Layers were separated and the aqueous layer was extracted twice more with diethyl ether. Combined organics were washed with brine, dried over Na₂SO₄, filtered and concentrated under reduced pressure, affording 2-methylbicyclo[2.2.1]heptan-2-ol (14.50 g, 115 mmol, 84.4%) as yellow oil. GCMS: Method 8, 1.97 min, 126.1, calcd. 126.2; ¹H NMR (400 MHz, CDCl₃) δ 2.19 (td, J=4.5, 2.8 Hz, 1H), 2.00 (dd, J=3.4, 1.7 Hz, 1H), 1.94 (dddd, J=12.2, 9.4, 5.1, 2.7 Hz, 1H), 1.63-1.48 (m, 3H), 1.37 (dt, J=12.1, 4.4 Hz, 1H), 1.32-1.26 (m, 6H), 1.17 (dd, J=12.7, 3.4 Hz, 1H).

Intermediate E: 5′H-spiro [bicyclo[2.2.1]heptane-2,2′-furan]

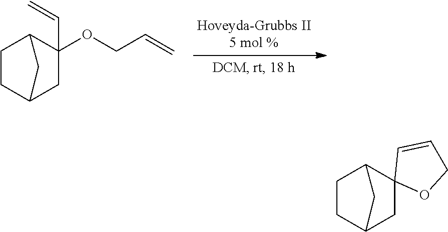

A 2-necked flask equipped with an outlet was dried under vacuum with a heat gun and then let it cool down to room temperature under argon flow. Then, DCM (0.460 L) previously degassed for 30 min, was added to the flask. A solution of 2-(allyloxy)-2-vinylbicyclo[2.2.1]heptane (2.5 g, 85% Wt, 1 Eq, 12 mmol) in degassed DCM (20 mL) was added to the reacting mixture, followed by a solution of dichloro(1,3-dimesityl-2-imidazolidinylidene)(2-isopropoxybenzylidene)ruthenium (0.37 g, 0.05 Eq, 0.60 mmol) in DCM (20 mL). The resulting solution was stirred at room temperature for 18 h. After reaction completion, the mixture was quenched by adding ethyl vinyl ether (0.43 g, 0.57 mL, 0.5 Eq, 6.0 mmol) (10 eq in respect of the catalyst) and the mixture was stirred at room temperature for 30 min. Then, the mixture was evaporated under reduced pressure affording a crude oil. The crude was purified via flash column chromatography (SiO₂, 0-10% MTBE in heptane), affording 5′H-spiro [bicyclo[2.2.1]heptane-2,2′-furan] (1.5 g, 8.8 mmol, 74% yield) as a colorless oil (complex mixture of isomers). Note: fast handling and storage under argon required due to moisture instability. GCMS, Method 8, 2.09 min, 150.1, calcd. 150.2; ¹H NMR (400 MHz, CDCl₃) δ 5.78-5.73 (m, 2H), 4.58 (dd, J=11.9, 1.6 Hz, 1H), 4.55-4.50 (m, 1H), 2.24 (s, 1H), 2.03 (dtd, J=9.5, 4.9, 2.6 Hz, 2H), 1.66 (ddd, J=12.9, 4.7, 2.9 Hz, 1H), 1.60-1.52 (m, 1H), 1.47 (dp, J=10.1, 2.0 Hz, 1H), 1.42-1.28 (m, 4H).

### Compounds of the Disclosure

### Example 1: 2-Vinylbicyclo[2.2.1]Hept-5-En-2-Ol

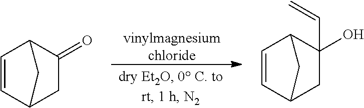

A stirring solution of bicyclo[2.2.1]hept-5-en-2-one (4.00 g, 1 Eq, 37.0 mmol) in dry diethyl ether (75 mL) was purged with nitrogen and cooled down to 0° C. Then, vinyl magnesium chloride in diethyl ether (49.3 mL, 1.5 molar, 2 Eq, 74.0 mmol) was added dropwise. After addition, the reaction mixture was allowed to warm to room temperature stirred for 1h. The reaction mixture was then cooled down on ice and quenched with aq. sat. NH₄Cl and layers were separated. The aqueous layer was extracted twice more with diethyl ether and combined organics were washed with brine, dried over Na₂SO₄, filtered and evaporated under reduced pressure affording a crude oil. The crude was purified by flash column chromatography (SiO₂, 0-100% EtOAc in heptane), affording 2-vinylbicyclo[2.2.1]hept-5-en-2-ol (3.68 g, 27.0 mmol, 73% yield) as faint yellow oil (98:2 mixture of isomers). GCMS: Method 8, 2.08 min, 136.1, calcd. 136.1; LCMS: Method 1, 1.1 min, M+H=119 [fragment mass]; calcd. 137.194. ¹H NMR (400 MHz, CDCl₃) δ 6.49 (dd, J=5.7, 3.0 Hz, 1H), 6.25-6.11 (m, 2H), 5.34 (dd, J=17.3, 1.3 Hz, 1H), 5.10 (dd, J=10.8, 1.4 Hz, 1H), 2.91 (q, J=2.3 Hz, 1H), 2.77 (dp, J=3.0, 1.5 Hz, 1H), 2.04 (dd, J=12.6, 3.8 Hz, 1H), 1.57-1.54 (m, 3H), 1.16 (dt, J=12.6, 2.0 Hz, 1H).

### Example 2: 2-Allylbicyclo[2.2.1]Hept-5-En-2-Ol

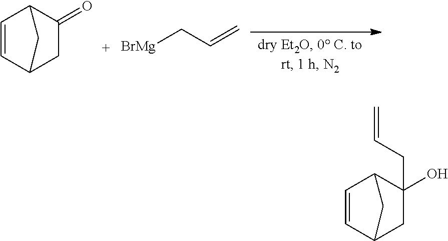

A stirring solution of bicyclo[2.2.1]hept-5-en-2-one (5.00 g, 1 Eq, 46.2 mmol) in dry diethyl ether (75 mL) was purged with nitrogen and then cooled down to 0° C. Then, allyl magnesium bromide in diethyl ether (92.5 mL, 1.0 molar, 2 Eq, 92.5 mmol) was added dropwise. After addition, the mixture was allowed to warm to room temperature and stir for 1h. The reaction mixture was then cooled down on ice and quenched with aq. sat. NH₄Cl and layers were separated. The aqueous layer was extracted twice more with diethyl ether and combined organics were washed with brine, dried over Na₂SO₄, filtered and evaporated under reduced pressure to give a crude oil. The residue was purified by flash column chromatography (SiO₂, 0-100% EtOAc in heptane), affording 2-allylbicyclo[2.2.1]hept-5-en-2-ol (4.91 g, 32.7 mmol, 71% yield) as light yellow oil (95:5 mixture of isomers). GCMS: Method 8, 2.18 and 2.20 min, 149.9 and 150.0, calcd. 150.1; LCMS: Method 5, 1.26 and 1.34 min, M+H=0; calcd. 151.221; ¹H NMR (400 MHz, CDCl₃) δ 6.44 (dd, J=5.8, 3.1 Hz, 1H), 6.18 (dd, J=5.8, 3.1 Hz, 1H), 6.07-5.95 (m, 1H), 5.17 (d, J=1.4 Hz, 1H), 5.15-5.12 (m, 1H), 2.85 (d, J=3.3 Hz, 1H), 2.77 (dq, J=4.1, 1.6 Hz, 1H), 2.54-2.50 (m, 2H), 1.86 (dd, J=12.4, 3.7 Hz, 1H), 1.60-1.55 (m, 2H), 1.53-1.49 (m, 1H), 1.10 (dd, J=12.4, 3.4 Hz, 1H).

### Example 3: 2-Allylbicyclo[2.2.1]Heptan-2-Ol

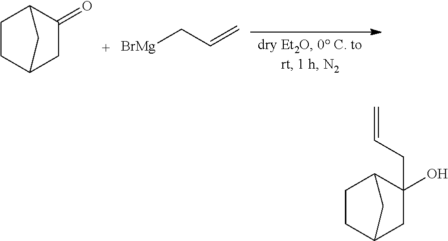

A stirring solution of bicyclo[2.2.1]heptan-2-one (5.00 g, 1 Eq, 45.4 mmol) in dry diethyl ether (75 mL) was purged with nitrogen and cooled down to 0° C. Allyl magnesium bromide in diethyl ether (90.8 mL, 1.0 molar, 2 Eq, 90.8 mmol) was added dropwise. After addition, the solution was allowed to warm to room temperature and stirred for 1h. The reaction mixture was cooled on ice and quenched by addition of aq. sat. NH₄Cl and then diluted with diethyl ether. Layers were separated and the aqueous layer was extracted thrice more with diethyl ether. Combined organics were dried over Na₂SO₄, filtered and evaporated under reduced pressure, affording a crude oil. The mixture was purified by flash column chromatography (SiO₂, 0-50% EtOAc in heptane) affording 2-allylbicyclo[2.2.1]heptan-2-ol (5.88 g, 38.6 mmol, 85% yield) as colorless oil. GCMS: Method 8, 2.25 min, 152.0, calcd. 152.2; ¹H NMR (400 MHz, CDCl₃) δ 5.91 (ddt, J=17.4, 10.3, 7.3 Hz, 1H), 5.23-5.10 (m, 2H), 2.39-2.22 (m, 2H), 2.23-2.18 (m, 1H), 2.10 (dd, J=3.9, 1.6 Hz, 1H), 1.98 (ddt, J=13.4, 10.3, 3.6 Hz, 1H), 1.62 (dddd, J=14.0, 9.9, 4.4, 2.0 Hz, 2H), 1.54-1.48 (m, 1H), 1.39-1.25 (m, 3H), 1.12 (dd, J=12.8, 3.3 Hz, 1H). OH proton not observed.

### Example 4: 2-Vinylbicyclo[2.2.1]Heptan-2-Ol

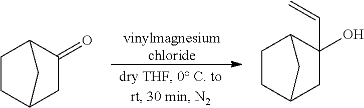

A stirring solution of bicyclo[2.2.1]heptan-2-one (5.00 g, 1 Eq, 45.4 mmol) in dry THF (75 mL) was purged with nitrogen and cooled down to 0° C. Vinyl magnesium chloride in THE (60.5 mL, 1.5 molar, 2 Eq, 90.8 mmol) was added dropwise. After addition, the solution was allowed to warm to room temperature and stirred for 30 min. The reaction mixture was cooled on ice and quenched by addition of aq. sat. NH₄Cl and then diluted with DCM. Layers were separated and the aqueous layer was extracted thrice more with DCM. Combined organics were dried over Na₂SO₄, filtered and concentrated under reduced pressure, affording a crude oil. The mixture was purified by flash column chromatography (SiO₂, 0-50% EtOAc in heptane), affording 2-vinylbicyclo[2.2.1]heptan-2-ol (5.01 g, 36.2 mmol, 79.9%) as a colorless oil. GCMS: Method 8, 2.11 min, 138.1, calcd. 138.2; ¹H NMR (400 MHz, CDCl₃) δ 6.05 (dd, J=17.3, 10.7 Hz, 1H), 5.17 (dd, J=17.2, 1.2 Hz, 1H), 5.00 (dd, J=10.6, 1.1 Hz, 1H), 2.24 (td, J=4.4, 2.6 Hz, 1H), 2.15-2.09 (m, 1H), 2.03 (tdt, J=8.7, 4.3, 1.8 Hz, 1H), 1.86 (ddd, J=13.0, 4.7, 2.8 Hz, 1H), 1.61-1.52 (m, 2H), 1.43-1.33 (m, 2H), 1.29 (ddt, J=10.1, 3.4, 1.7 Hz, 1H), 1.18 (dd, J=13.0, 3.4 Hz, 1H). OH proton not observed.

### Example 5: Bicyclo[2.2.1]Hept-5-En-2-Yl Isobutyrate

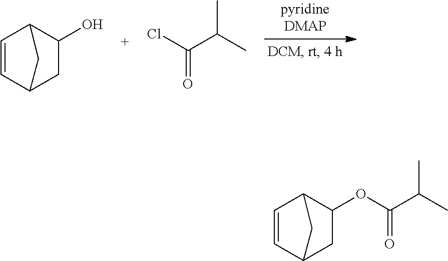

To a stirring solution of bicyclo[2.2.1]hept-5-en-2-ol (800 mg, 1 Eq, 7.26 mmol) in DCM (8.0 mL) was added Isobutyryl chloride (1.14 mL, 1.5 Eq, 10.9 mmol), pyridine (1.76 mL, 3.0 Eq, 21.8 mmol) and DMAP (177 mg, 0.2 Eq, 1.45 mmol). The reaction mixture was stirred for 4h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was purified by flash column chromatography (SiO₂, 0-20% MTBE in heptane), affording bicyclo[2.2.1]hept-5-en-2-yl isobutyrate (460 mg, 2.55 mmol, 35% yield) as a colorless oil (95:5 mixture of isomers). ¹H NMR (400 MHz, CDCl₃) δ 6.33 (dd, J=5.8, 3.1 Hz, 1H), 5.95 (dd, J=5.7, 2.9 Hz, 1H), 5.26 (ddd, J=8.2, 3.9, 2.8 Hz, 1H), 3.14 (tt, J=3.7, 1.9 Hz, 1H), 2.84 (tt, J=3.2, 1.5 Hz, 1H), 2.48-2.41 (m, 1H), 2.13 (ddd, J=12.2, 8.2, 3.7 Hz, 1H), 1.46 (ddt, J=9.0, 3.9, 2.0 Hz, 1H), 1.34-1.31 (m, 1H), 1.11 (dd, J=7.0, 5.2 Hz, 6H), 0.90 (dt, J=12.5, 3.3 Hz, 1H).

### Example 6: Bicyclo[2.2.1]Hept-5-En-2-Yl Benzoate

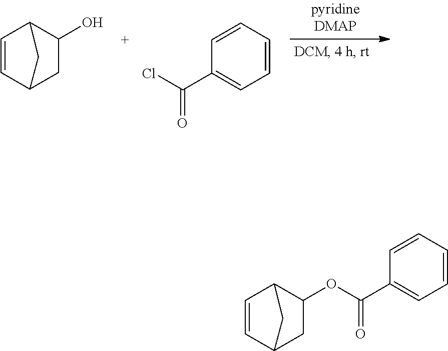 [SMILES: OC1CC2C=CC1C2 ; O=C(Cl)c1ccccc1 ; O=C(OC1CC2C=CC1C2)c1ccccc1]

To a stirring solution of bicyclo[2.2.1]hept-5-en-2-ol (800 mg, 1 Eq, 7.26 mmol) in DCM (8.0 mL) was added benzoyl chloride (1.26 mL, 1.5 Eq, 10.9 mmol), pyridine (1.76 mL, 3.0 Eq, 21.8 mmol) and DMAP (177 mg, 0.2 Eq, 1.45 mmol). The reaction mixture was stirred for 4h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was purified by flash column chromatography (SiO₂, 0-20% MTBE in heptane), affording bicyclo[2.2.1]hept-5-en-2-yl benzoate (1.0 g, 4.67 mmol, 64% yield) as a colorless oil (95:5 mixture of isomers). LCMS: Method 5, 1.87 min, M+H=186.0 [fragment mass], calcd. 215.264; ¹H NMR (400 MHz, CDCl₃) δ 7.99-7.93 (m, 2H), 7.56-7.51 (m, 1H), 7.41 (dd, J=8.4, 7.1 Hz, 2H), 6.38 (dd, J=5.8, 3.0 Hz, 1H), 6.07 (dd, J=5.8, 2.9 Hz, 1H), 5.53 (ddd, J=8.1, 3.9, 2.8 Hz, 1H), 3.27 (q, J=2.1 Hz, 1H), 2.91 (dq, J=3.6, 1.8 Hz, 1H), 2.25 (ddd, J=12.1, 8.1, 3.7 Hz, 1H), 1.53 (ddt, J=9.0, 3.9, 2.0 Hz, 1H), 1.44-1.37 (m, 1H), 1.09 (dt, J=12.6, 3.3 Hz, 1H).

### Example 7: Bicyclo[2.2.1]Hept-5-En-2-Yl Acetate

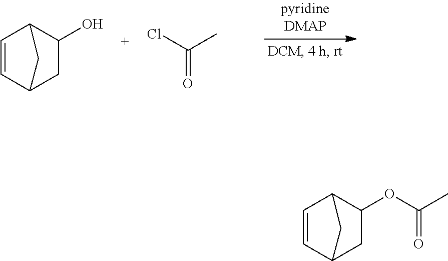

To a stirring solution of bicyclo[2.2.1]hept-5-en-2-ol (800 mg, 1 Eq, 7.26 mmol) in DCM (8.0 mL) was added acetyl chloride (775 μL, 1.5 Eq, 10.9 mmol), pyridine (1.76 mL, 3.0 Eq, 21.8 mmol) and DMAP (177 mg, 0.2 Eq, 1.45 mmol). The reaction mixture was stirred for 4h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was purified by flash column chromatography (SiO₂, 0-20% MTBE in heptane), affording bicyclo[2.2.1]hept-5-en-2-yl acetate (340 mg, 2.23 mmol, 31% yield) as a colorless oil. ¹H NMR (400 MHz, CDCl₃) δ 6.34 (dd, J=5.7, 3.1 Hz, 1H), 5.97 (dd, J=5.8, 2.9 Hz, 1H), 5.30-5.24 (m, 1H), 3.13 (dp, J=3.7, 1.9 Hz, 1H), 2.84 (dq, J=3.7, 1.9 Hz, 1H), 2.14 (ddd, J=12.2, 8.2, 3.7 Hz, 1H), 1.98 (s, 3H), 1.47 (ddt, J=9.0, 3.9, 2.0 Hz, 1H), 1.34-1.30 (m, 1H), 0.93 (dt, J=12.6, 3.3 Hz, 1H).

### Example 8: Bicyclo[2.2.1]Hept-5-En-2-Yl Propionate

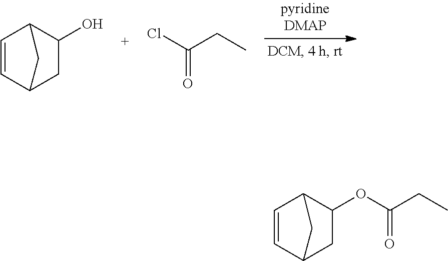

To a stirring solution of bicyclo[2.2.1]hept-5-en-2-ol (800 mg, 1 Eq, 7.26 mmol) in DCM (8.0 mL) was added propionyl chloride (0.950 mL, 1.5 Eq, 10.9 mmol), pyridine (1.76 mL, 3.0 Eq, 21.8 mmol) and DMAP (177 mg, 0.2 Eq, 1.45 mmol). The reaction mixture was stirred for 4h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was purified by flash column chromatography (SiO₂, 0-20% MTBE in heptane), affording bicyclo[2.2.1]hept-5-en-2-yl propionate (720 mg, 4.33 mmol, 60% yield) as a colorless oil (95:5 mixture of isomers). GCMS: Method 8, 2.27 min, 166.1, calcd. 166.2; ¹H NMR (400 MHz, CDCl₃) δ 6.33 (dd, J=5.7, 3.1 Hz, 1H), 5.96 (dd, J=5.7, 2.9 Hz, 1H), 5.28 (ddd, J=8.2, 3.9, 2.8 Hz, 1H), 3.14 (tt, J=3.6, 1.9 Hz, 1H), 2.84 (tt, J=3.4, 1.6 Hz, 1H), 2.24 (q, J=7.6 Hz, 2H), 2.14 (ddd, J=12.2, 8.2, 3.7 Hz, 1H), 1.46 (ddt, J=9.0, 3.9, 2.0 Hz, 1H), 1.34-1.29 (m, 1H), 1.09 (t, J=7.6 Hz, 3H), 0.92 (dt, J=12.5, 2.9 Hz, 1H).

### Example 9: Bicyclo[2.2.1]Hept-5-En-2-Yl Butyrate

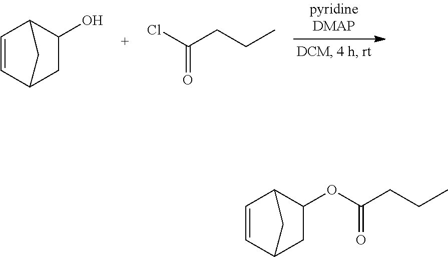

To a stirring solution of bicyclo[2.2.1]hept-5-en-2-ol (800 mg, 1 Eq, 7.26 mmol) in DCM (8.0 mL) was added n-butyryl chloride (1.13 mL, 1.5 Eq, 10.9 mmol), pyridine (1.76 mL, 3.0 Eq, 21.8 mmol) and DMAP (177 mg, 0.2 Eq, 1.45 mmol). The reaction mixture was stirred for 4h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was first dissolved in little DCM and filtered over silica, washing the filter cake with 5% EtOAc in heptane solution. The filtrate was then evaporated under reduced pressure and purified by flash column chromatography (SiO₂, 0-20% MTBE in heptane), affording bicyclo[2.2.1]hept-5-en-2-yl butyrate (400 mg, 2.22 mmol, 31% yield) as a colorless oil (95:5 mixture of isomers). GCMS: Method 8, 2.40 min, 180.1, calcd. 180.2; ¹H NMR (400 MHz, CDCl₃) δ 6.34 (dt, J=5.8, 3.2 Hz, 1H), 5.96 (dd, J=5.7, 2.9 Hz, 1H), 5.28 (dq, J=7.2, 3.2 Hz, 1H), 3.14 (s, 1H), 2.84 (s, 1H), 2.21 (td, J=7.5, 3.0 Hz, 2H), 2.14 (ddd, J=12.2, 8.1, 3.7 Hz, 1H), 1.68-1.60 (m, 2H), 1.47 (ddq, J=8.7, 3.8, 2.2 Hz, 1H), 1.33 (m, 1H), 0.95-0.90 (m, 4H).

### Example 10: Bicyclo[2.2.1]Hept-5-En-2-Yl (E)-But-2-Enoate

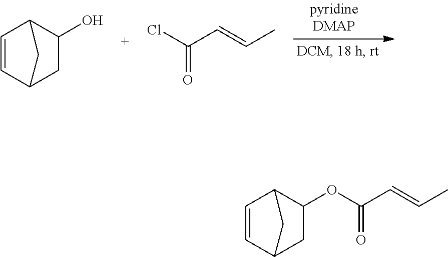

To a stirring solution of bicyclo[2.2.1]hept-5-en-2-ol (1.00 g, 1 Eq, 9.08 mmol) in DCM (10.0 mL) was added (E)-but-2-enoyl chloride (1.42 g, 1.31 mL, 1.5 Eq, 13.6 mmol), pyridine (2.15 g, 2.20 mL, 3 Eq, 27.2 mmol) and DMAP (222 mg, 0.2 Eq, 1.82 mmol). The reaction mixture was stirred for 18h at room temperature. The reacting mixture was then diluted with DCM and washed thrice with water. The aqueous layer was collected and saturated with some brine then extracted back with fresh DCM. Combined organic layers were washed with brine, dried over Na₂SO₄, filtered and concentrated under reduced pressure. The crude was purified by flash column chromatography (SiO₂, 0-30% diethyl ether in pentane), affording bicyclo[2.2.1]hept-5-en-2-yl (E)-but-2-enoate (365.20 mg, 2.0490 mmol, 22.6%) as a colorless oil. GCMS: Method 8, 2.47 min, 178.1, calcd. 178.2; LCMS: Method 2, 1.67 min, M+H=179.2; calcd. 179.231. ¹H NMR (400 MHz, CDCl₃) δ 6.90 (dq, J=15.6, 6.9 Hz, 1H), 6.34 (dd, J=5.8, 3.0 Hz, 1H), 5.98 (dd, J=5.7, 2.9 Hz, 1H), 5.77 (dq, J=15.5, 1.7 Hz, 1H), 5.33 (dt, J=8.2, 3.3 Hz, 1H), 3.19-3.12 (m, 1H), 2.85 (dq, J=3.5, 1.8 Hz, 1H), 2.16 (ddd, J=12.2, 8.2, 3.7 Hz, 1H), 1.85 (dd, J=6.8, 1.6, 3H), 1.47 (ddt, J=9.0, 3.9, 2.0 Hz, 1H), 1.34 (d, J=8.8 Hz, 1H), 0.95 (dt, J=12.5, 3.3 Hz, 1H).

### Example 11: Bicyclo[2.2.1]Hept-5-En-2-Yl Pivalate

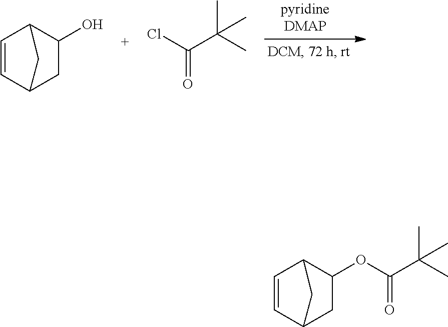

To bicyclo[2.2.1]hept-5-en-2-ol (1.00 g, 1 Eq, 9.08 mmol) in DCM (20 mL) were added DMAP (222 mg, 0.2 Eq, 1.82 mmol), pyridine (2.20 mL, 3 Eq, 27.2 mmol) and trimethylacetyl chloride (13.34 mL, 3.0 Eq, 27.2 mmol). The reaction mixture was stirred for 72 h at room temperature. The reacting mixture was then diluted with DCM and washed thrice with water. The aqueous layer was collected and extracted back twice with DCM. Combined organic layers were washed with brine, dried over Na₂SO₄, filtered and concentrated under reduced pressure. The crude was purified twice by flash column chromatography (SiO₂, 0-30% diethyl ether in pentane and then 0-20% MTBE in heptane) affording bicyclo[2.2.1]hept-5-en-2-yl pivalate (225 mg, 1.16mmol, 13% yield) as a colorless oil. GCMS: Method 8, 2.35 min, 194.1, calcd. 194.2; ¹H NMR (400 MHz, CDCl₃) δ 6.31 (dd, J=5.8, 3.0 Hz, 1H), 5.94 (dd, J=5.7, 2.9 Hz, 1H), 5.23 (ddd, J=8.2, 4.0, 2.8 Hz, 1H), 3.13 (tt, J=3.5, 1.8 Hz, 1H), 2.84 (dp, J=5.2, 2.0 Hz, 1H), 2.12 (ddd, J=12.1, 8.1, 3.7 Hz, 1H), 1.46 (ddt, J=9.0, 3.8, 2.0 Hz, 1H), 1.35-1.30 (m, 1H), 1.13 (s, 9H), 0.88 (dt, J=12.4, 3.2 Hz, 1H).

### Example 12: Bicyclo[2.2.1]Hept-5-En-2-Yl (E)-2-Methylbut-2-Enoate

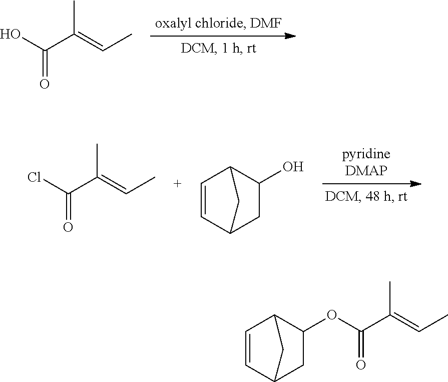

Tiglic acid (3.63 mL, 1 Eq, 35 mmol) was dissolved in DCM (50 mL) and oxalyl chloride (4.6 mL, 1.5 Eq, 52 mmol) and 1 drop of DMF were added. The reaction mixture was stirred at room temperature for 1h. After full conversion of the acid was achieved, the reaction mixture was stopped and concentrated under reduced pressure affording (E)-2-methylbut-2-enoyl chloride as a brown residue. Part of the crude residue was used as such in the next step. To bicyclo[2.2.1]hept-5-en-2-ol (1.00 g, 1 Eq, 9.08 mmol) in DCM (20 mL) was added (E)-2-methylbut-2-enoyl chloride (1.6 g, 1.5 Eq, 13.6 mmol), pyridine (2.20 mL, 3 Eq, 27.2 mmol) and DMAP (222 mg, 0.2 Eq, 1.82 mmol) and mixtures stirred at rt for 48h. After reaction completion, the mixture was diluted with water and filtered off over a phase separator. The filtrate was first concentrated under reduced pressure and then purified by flash column chromatography (SiO₂, 0-30% diethyl ether in pentane), affording bicyclo[2.2.1]hept-5-en-2-yl (E)-2-methylbut-2-enoate (344 mg, 1.8 mmol, 19.7%) as a colorless oil. GCMS: Method 8, 2.61 min, 192.1, calcd. 192.2; LCMS: Method 5, 1.82 min, M+H=193.1; calcd. 193.258. ¹H NMR (400 MHz, CDCl₃) δ 6.73 (qd, J=7.0, 1.2 Hz, 1H), 6.33 (dd, J=5.8, 3.0 Hz, 1H), 5.99 (dd, J=5.8, 2.9 Hz, 1H), 5.32 (dt, J=8.1, 3.2 Hz, 1H), 3.16 (dq, J=3.3, 1.9 Hz, 1H), 2.85 (td, J=3.5, 1.8 Hz, 1H), 2.16 (ddd, J=12.1, 8.1, 3.7 Hz, 1H), 1.81-1.72 (m, 6H), 1.47 (ddt, J=8.9, 4.0, 2.0 Hz, 1H), 1.34 (d, J=8.9 Hz, 1H), 0.95 (dt, J=12.5, 3.3 Hz, 1H).

### Example 13: Bicyclo[2.2.1]Hept-5-En-2-Yl (E)-2-Methylpent-2-Enoate

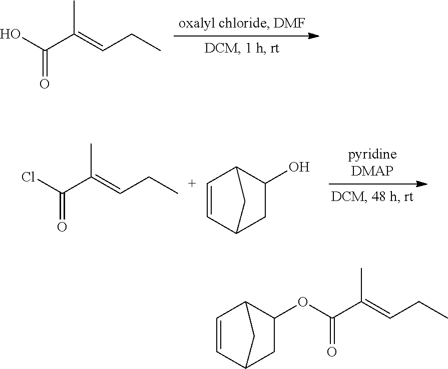

(E)-2-methylpent-2-enoic acid (3.5 g, 1 Eq, 31 mmol) was dissolved in DCM (50 mL) and oxalyl chloride (4.0 mL, 1.5 Eq, 46 mmol) and 1 drop of DMF were added. The reaction mixture was stirred at room temperature for 1h. After full conversion of the acid was achieved, the reaction mixture was stopped and concentrated under reduced pressure affording (E)-2-methylpent-2-enoyl chloride as a brown residue. Part of the crude residue was used as such in the next step. To bicyclo[2.2.1]hept-5-en-2-ol (1.00 g, 1 Eq, 9.08 mmol) in DCM (20 mL) was added (E)-2-methylpent-2-enoyl chloride (1.8 g, 1.5 Eq, 13.6 mmol), pyridine (2.20 mL, 3 Eq, 27.2 mmol) and DMAP (222 mg, 0.2 Eq, 1.82 mmol) and mixtures stirred at rt for 48h. After reaction completion, the mixture was diluted with water and filtered off over a phase separator. The filtrate was first concentrated under reduced pressure and then purified by flash column chromatography (SiO₂, 0-30% diethyl ether in pentane), affording bicyclo[2.2.1]hept-5-en-2-yl (E)-2-methylpent-2-enoate (404.5 mg, 1.96 mmol, 22% yield) as a colorless oil. GCMS: Method 8, 2.72 min, 206.1, calcd. 206.3; LCMS: Method 5, 1.94 min, M+H=207.2; calcd. 207.285; ¹H NMR (400 MHz, CDCl₃) δ 6.63 (tq, J=7.4, 1.5 Hz, 1H), 6.33 (dd, J=5.8, 3.0 Hz, 1H), 5.99 (dd, J=5.7, 3.0 Hz, 1H), 5.32 (ddd, J=8.2, 3.9, 2.8 Hz, 1H), 3.16 (tq, J=3.6, 1.7 Hz, 1H), 2.85 (dq, J=3.6, 1.9 Hz, 1H), 2.20-2.11 (m, 3H), 1.77 (d, J=1.2 Hz, 3H), 1.47 (ddt, J=9.0, 3.8, 2.0 Hz, 1H), 1.36-1.32 (m, 1H), 1.03 (t, J=7.6 Hz, 3H), 0.96 (dt, J=12.5, 3.3 Hz, 1H).

### Example 14: 5-Methoxybicyclo[2.2.1]Hept-2-Ene

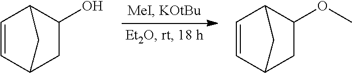

To bicyclo[2.2.1]hept-5-en-2-ol (1.00 g, 1 Eq, 9.08 mmol) in diethyl ether (40 mL) were added potassium tert-butoxide (2.04 g, 2 Eq, 18.2 mmol) iodomethane (0.85 mL, 1.5 Eq, 13.6 mmol). The reacting mixture was stirred at room temperature for 18 h. After reaction completion, the mixture was diluted with water and diethyl ether and layers were separated. The aqueous layer was extracted twice more with diethyl ether, then the organics were combined, washed once with brine, dried over Na₂SO₄, filtered and evaporated under reduced pressure. The crude was purified by flash column chromatography (SiO₂, 0-30% diethyl ether in pentane), affording 5-methoxybicyclo[2.2.1]hept-2-ene (434 mg, 3.50 mmol, 38% yield) as a colorless oil. GCMS: Method 8, 1.87 min, 124.1, calcd. 124.2; ¹H NMR (400 MHz, CDCl₃) δ 6.33 (dd, J=5.8, 3.0 Hz, 1H), 6.00 (dd, J=5.8, 3.0 Hz, 1H), 4.06 (dt, J=8.0, 3.2 Hz, 1H), 3.29 (s, 3H), 3.11 (tt, J=3.6, 1.7 Hz, 1H), 2.80 (td, J=3.5, 1.9 Hz, 1H), 1.98 (ddd, J=11.9, 7.9, 3.8 Hz, 1H), 1.45 (ddt, J=8.8, 3.9, 2.0 Hz, 1H), 1.24 (dd, J=8.7, 1.5 Hz, 1H), 0.88 (dt, J=12.2, 3.3 Hz, 1H).

### Example 15: -(Allyloxy)Bicyclo[2.2.1]Hept-2-Ene

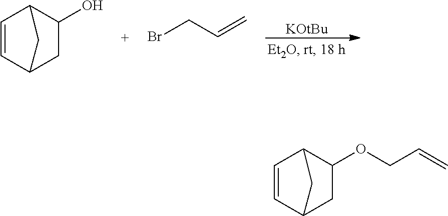

To bicyclo[2.2.1]hept-5-en-2-ol (1.00 g, 1 Eq, 9.08 mmol) in diethyl ether (40 mL) were added potassium tert-butoxide (2.04 g, 2 Eq, 18.2 mmol) and allyl bromide (1.65 g, 1.17 mL, 1.5 Eq, 13.6 mmol). The reacting mixture was stirred at room temperature for 18 h. After reaction completion, the mixture was diluted with water and diethyl ether and layers were separated. The aqueous layer was extracted twice more with diethyl ether, then the organics were combined, washed once with brine, dried over Na₂SO₄, filtered and evaporated under reduced pressure. The crude was purified by flash column chromatography (SiO₂, 0-30% diethyl ether in pentane), affording 5-(allyloxy)bicyclo[2.2.1]hept-2-ene (421 mg, 2.80 mmol, 31% yield). GCMS: Method 8, 2.12 min, 150.0, calcd. 150.2; ¹H NMR (400 MHz, CDCl₃) δ 6.33 (dd, J=5.8, 3.1 Hz, 1H), 5.99 (dd, J=5.8, 2.9 Hz, 1H), 5.97-5.85 (m, 1H), 5.26 (dq, J=17.1, 1.8 Hz, 1H), 5.15 (dq, J=10.4, 1.5 Hz, 1H), 4.20 (dt, J=8.0, 3.3 Hz, 1H), 4.01-3.89 (m, 2H), 3.09 (dq, J=3.5, 1.9 Hz, 1H), 2.79 (q, J=2.8 Hz, 1H), 1.98 (ddd, J=11.9, 8.0, 3.8 Hz, 1H), 1.43 (ddt, J=8.2, 3.9, 2.0 Hz, 1H), 1.23 (d, J=8.8 Hz, 1H), 0.91 (dt, J=12.2, 3.4 Hz, 1H).

### Example 16: 5-((2-Methylallyl)Oxy)Bicyclo[2.2.1]Hept-2-Ene

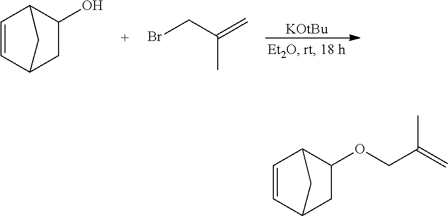

To bicyclo[2.2.1]hept-5-en-2-ol (1.00 g, 1 Eq, 9.08 mmol) in diethyl ether (40 mL) were added potassium tert-butoxide (2.04 g, 2 Eq, 18.2 mmol) and 3-Bromoisobutylene (1.37 mL, 1.5 Eq, 13.6 mmol). The reacting mixture was stirred at room temperature for 18 h. After reaction completion, the mixture was diluted with water and diethyl ether and layers were separated. The aqueous layer was extracted twice more with diethyl ether, then the organics were combined, washed once with brine, dried over Na₂SO₄, filtered and evaporated under reduced pressure. The crude was purified by flash column chromatography (SiO₂, 0-30% diethyl ether in pentane), affording 5-((2-methylallyl)oxy)bicyclo[2.2.1]hept-2-ene (302 mg, 1.84 mmol, 20% yield). GCMS: Method 8, 2.23 min, 164.0, calcd. 164.2; ¹H NMR (400 MHz, CDCl₃) δ 6.32 (dd, J=5.8, 3.0 Hz, 1H), 6.00 (dd, J=5.8, 2.9 Hz, 1H), 4.95 (dd, J=2.2, 1.2 Hz, 1H), 4.86 (d, J=1.9 Hz, 1H), 4.17 (dt, J=8.0, 3.2 Hz, 1H), 3.91-3.79 (m, 2H), 3.08 (dp, J=3.5, 1.7 Hz, 1H), 2.79 (dq, J=3.7, 1.9 Hz, 1H), 1.96 (ddd, J=11.9, 7.9, 3.8 Hz, 1H), 1.72 (s, 3H), 1.42 (ddt, J=8.0, 4.0, 2.0 Hz, 1H), 1.27-1.20 (m, 1H), 0.91 (dt, J=12.0, 3.3 Hz, 1H).

### Example 17: 5-Ethoxybicyclo[2.2.1]Hept-2-Ene

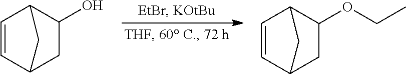

To a stirring solution of bicyclo[2.2.1]hept-5-en-2-ol (750 mg, 1 Eq, 6.81 mmol) in THF (10.0 mL) was added potassium tert-butoxide (1.15 g, 1.5 Eq, 10.2 mmol). Then ethyl bromide (762 μL, 1.5 Eq, 10.2 mmol) was added and the reaction mixture was stirred at 60° C. for 72h. After reaction completion, the reacting mixture was stopped and allowed to cool down to room temperature. The content of the flask was evaporated and purified by flash column chromatography (SiO₂, 0-20% MTBE in heptane), affording 5-ethoxybicyclo[2.2.1]hept-2-ene (240 mg, 1.74 mmol, 25% yield) as a colorless oil. GCMS: Method 8, 1.96 min, 138.0, calcd. 138.2; ¹H NMR (400 MHz, CDCl₃) δ 6.33 (dd, J=5.7, 3.0 Hz, 1H), 5.98 (dd, J=5.7, 2.9 Hz, 1H), 4.17-4.10 (m, 1H), 3.52 (dq, J=9.4, 7.0 Hz, 1H), 3.42 (dq, J=9.4, 7.1 Hz, 1H), 3.13-3.06 (m, 1H), 2.79 (d, J=4.2 Hz, 1H), 1.98 (ddd, J=11.9, 8.0, 3.8 Hz, 1H), 1.43 (ddt, J=8.8, 3.8, 2.0 Hz, 1H), 1.28-1.22 (m, 1H), 1.17 (t, J=7.0 Hz, 3H), 0.88 (dt, J=12.0, 3.2 Hz, 1H).

### Example 18: 2-Methylbicyclo[2.2.1]Hept-5-En-2-Yl Benzoate

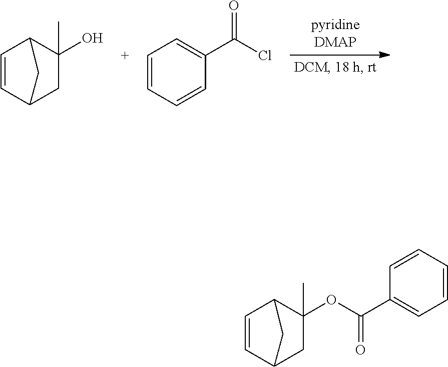

To a stirring solution of 2-methylbicyclo[2.2.1]hept-5-en-2-ol (750 mg, 1 Eq, 6.04 mmol) in DCM (10.0 mL) was added benzoyl chloride (934 mg, 0.77 mL, 1.1 Eq, 6.64 mmol), pyridine (1.47 mL, 3 Eq, 18.1 mmol) and DMAP (148 mg, 0.2 Eq, 1.21 mmol). The reaction mixture was stirred for 18 h at room temperature. The reaction was then concentrated under reduced pressure and the resulting crude residue was purified by flash column chromatography (SiO₂, 0-20% MTBE in heptane), affording 2-methylbicyclo[2.2.1]hept-5-en-2-yl benzoate (770 mg, 3.37 mmol, 56% yield) as a colorless oil. GCMS: Method 8, 3.00 min, 228.1, calcd. 228.3; LCMS: Method 5, 1.96 min, M+H=229.0; calcd. 229.291; ¹H NMR (400 MHz, CDCl₃) δ 7.94-7.89 (m, 2H), 7.53-7.48 (m, 1H), 7.40 (dd, J=8.3, 7.0 Hz, 2H), 6.26 (dd, J=5.7, 3.0 Hz, 1H), 6.08 (dd, J=5.7, 3.0 Hz, 1H), 3.33 (dq, J=3.3, 1.7 Hz, 1H), 2.87 (tq, J=3.2, 1.8 Hz, 1H), 1.93 (dd, J=12.8, 3.6 Hz, 1H), 1.82 (s, 3H), 1.73 (dd, J=12.8, 3.5 Hz, 1H), 1.58 (ddt, J=9.0, 3.5, 1.8 Hz, 1H), 1.54-1.51 (m, 1H).

### Example 19: 2-Methylbicyclo[2.2.1]Hept-5-En-2-Yl Isobutyrate

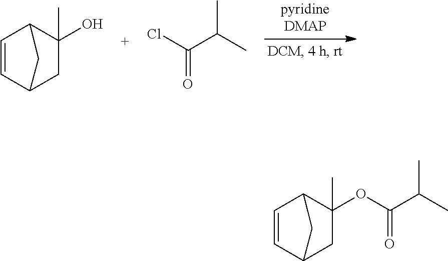

To a stirring solution of 2-methylbicyclo[2.2.1]hept-5-en-2-ol (850 mg, 1 Eq, 6.84 mmol) in DCM (10.0 mL) was added isobutyryl chloride (1.08 mL, 1.5 Eq, 10.3 mmol), pyridine (1.66 mL, 3.0 Eq, 20.5 mmol) and DMAP (167 mg, 0.2 Eq, 1.37 mmol). The reaction mixture was stirred for 4h at room temperature. The reacting mixture was then diluted with DCM and washed thrice with water. Combined organic layers were washed with brine, dried over Na₂SO₄, filtered and concentrated under reduced pressure. The crude was then purified by flash column chromatography (SiO₂, 0-20% MTBE in heptane), affording 2-methylbicyclo[2.2.1]hept-5-en-2-yl isobutyrate (106 mg, 0.55 mmol, 8% yield) as a light yellow oil (94:6 mixture of isomers). GCMS: Method 8, 2.35 min, 194.1, calcd. 194.2; LCMS: Method 5, 1.88 min, M+H=195.3; calcd. 195.274; ¹H NMR (400 MHz, CDCl₃) δ 6.22 (dd, J=5.7, 3.0 Hz, 1H), 5.98 (dd, J=5.8, 3.0 Hz, 1H), 3.21 (dt, J=3.2, 1.7 Hz, 1H), 2.81 (q, J=2.2 Hz, 1H), 2.36 (p, J=7.0 Hz, 1H), 1.83-1.76 (m, 1H), 1.67 (s, 3H), 1.54-1.48 (m, 2H), 1.46-1.42 (m, 1H), 1.07 (dd, J=7.0, 1.1 Hz, 6H).

### Example 20: 2-Methylbicyclo[2.2.1]Hept-5-En-2-Yl Acetate

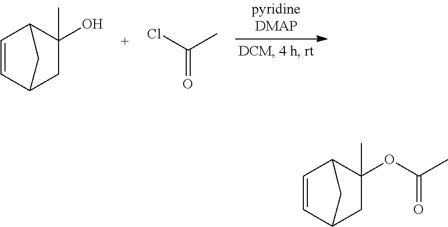

To a stirring solution of 2-methylbicyclo[2.2.1]hept-5-en-2-ol (850 mg, 1 Eq, 6.84 mmol) in DCM (10.0 mL) was added acetyl chloride (0.73 mL, 1.5 Eq, 10.3 mmol), pyridine (1.66 mL, 3.0 Eq, 20.5 mmol) and DMAP (167 mg, 0.2 Eq, 1.37 mmol). The reaction mixture was stirred for 4h at room temperature. The reacting mixture was then diluted with DCM and washed thrice with water. Combined organic layers were washed with brine, dried over Na₂SO₄, filtered and concentrated under reduced pressure. The crude was then purified by flash column chromatography (SiO₂, 0-20% MTBE in heptane), affording 2-methylbicyclo[2.2.1]hept-5-en-2-yl acetate (452 mg, 2.72 mmol, 40% yield) as a colorless oil (95:5 mixture of isomers). GCMS: Method 8, 2.16 min, 166.1, calcd. 166.2; ¹H NMR (400 MHz, CDCl₃) δ 6.24 (dd, J=5.7, 3.0 Hz, 1H), 6.00 (dd, J=5.8, 3.0 Hz, 1H), 3.20 (h, J=1.5 Hz, 1H), 2.81 (td, J=3.4, 1.7 Hz, 1H), 1.90 (s, 3H), 1.79 (dd, J=12.5, 3.6 Hz, 1H), 1.68 (s, 3H), 1.56-1.49 (m, 2H), 1.44 (dt, J=8.7, 1.6 Hz, 1H).

### Example 21: 2-Methylbicyclo[2.2.1]Hept-5-En-2-Yl Propionate

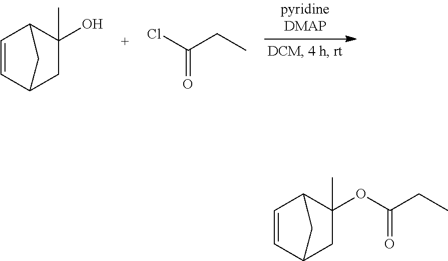

To a stirring solution of 2-methylbicyclo[2.2.1]hept-5-en-2-ol (850 mg, 1 Eq, 6.84 mmol) in DCM (10.0 mL) was added propionyl chloride (0.90 mL, 1.5 Eq, 10.3 mmol), pyridine (1.66 mL, 3.0 Eq, 20.5 mmol) and DMAP (167 mg, 0.2 Eq, 1.37 mmol). The reaction mixture was stirred for 4h at room temperature. The reacting mixture was then diluted with DCM and washed thrice with water. Combined organic layers were washed with brine, dried over Na₂SO₄, filtered and concentrated under reduced pressure. The crude was then purified by flash column chromatography (SiO₂, 0-20% MTBE in heptane), affording 2-methylbicyclo[2.2.1]hept-5-en-2-yl propionate (497 mg, 2.76 mmol, 40% yield) as a colorless oil. GCMS: Method 8, 2.28 min, 180.1, calcd. 180.1; ¹H NMR (400 MHz, CDCl₃) δ 6.23 (dd, J=5.7, 3.0 Hz, 1H), 5.98 (dd, J=5.7, 3.0 Hz, 1H), 3.21 (dd, J=3.0, 1.6 Hz, 1H), 2.81 (q, J=3.0 Hz, 1H), 2.16 (q, J=7.6 Hz, 2H), 1.79 (dd, J=12.5, 3.6 Hz, 1H), 1.68 (s, 3H), 1.52 (ddt, J=10.2, 3.6, 2.7 Hz, 2H), 1.46-1.42 (m, 1H), 1.06 (t, J=7.6 Hz, 3H).

### Example 22: 2-Methylbicyclo[2.2.1]Hept-5-En-2-Yl Butyrate

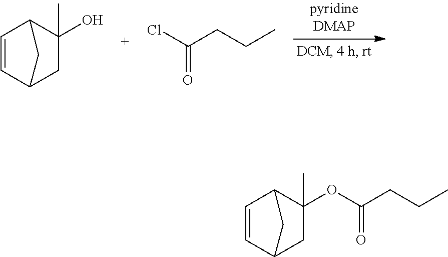

To a stirring solution of 2-methylbicyclo[2.2.1]hept-5-en-2-ol (850 mg, 1 Eq, 6.84 mmol) in DCM (10.0 mL) was added acetyl chloride (0.73 mL, 1.5 Eq, 10.3 mmol), pyridine (1.66 mL, 3.0 Eq, 20.5 mmol) and DMAP (167 mg, 0.2 Eq, 1.37 mmol). The reaction mixture was stirred for 4h at room temperature. The reacting mixture was then diluted with DCM and washed thrice with water. Combined organic layers were washed with brine, dried over Na₂SO₄, filtered and concentrated under reduced pressure. The crude was then purified by flash column chromatography (SiO₂, 0-20% MTBE in heptane), affording 2-methylbicyclo[2.2.1]hept-5-en-2-yl butyrate (402 mg, 2.07 mmol, 30% yield) as a colorless oil. GCMS: Method 8, 2.16 min, 166.1, calcd. 166.2; LCMS: Method 5, 1.872 min, M+H=195.3; calcd. 195.274; ¹H NMR (400 MHz, CDCl₃) δ 6.23 (dd, J=5.7, 3.0 Hz, 1H), 5.99 (dd, J=5.7, 3.0 Hz, 1H), 3.21 (h, J=1.4 Hz, 1H), 2.81 (d, J=4.1 Hz, 1H), 2.15-2.09 (m, 2H), 1.80 (dd, J=12.5, 3.6 Hz, 1H), 1.68 (s, 3H), 1.61-1.58 (m, 1H), 1.55-1.53 (m, 1H), 1.50 (ddt, J=5.3, 3.6, 2.0 Hz, 2H), 1.46-1.42 (m, 1H), 0.91 (t, J=7.4 Hz, 3H).

### Example 23: 2-Methylbicyclo[2.2.1]Hept-5-En-2-Yl (E)-But-2-Enoate

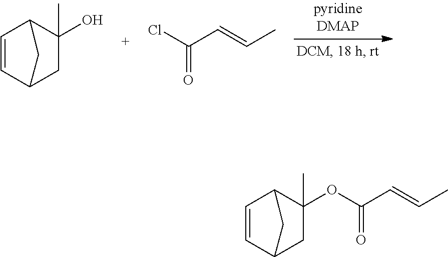

To a stirring solution of bicyclo[2.2.1]heptan-2-ol (1.0 g, 1 Eq, 8.1 mmol) in DCM (10.0 mL) was added (E)-crotonoyl chloride (1.2 mL, 1.5 Eq, 12.0 mmol), pyridine (2.0 mL, 3 Eq, 24 mmol) and DMAP (0.20 g, 0.2 Eq, 1.6 mmol). The reaction mixture was stirred for 18 h at room temperature. After reaction completion, the mixture was diluted with water and filtered off over a phase separator. The filtrate was first concentrated and reduced pressure and then purified by flash column chromatography (SiO₂, 0-20% MTBE in heptane), affording 2-methylbicyclo[2.2.1]hept-5-en-2-yl (E)-but-2-enoate (163 mg, 0.85 mmol, 11% yield) as a colorless oil. GCMS: Method 8, 2.48 min, 192.1, calcd. 192.3; LCMS: Method 2, 1.93 min, M+H=193.2; calcd. 193.258; ¹H NMR (400 MHz, CDCl₃) δ 6.82 (dq, J=15.7, 6.9 Hz, 1H), 6.23 (dd, J=5.7, 3.0 Hz, 1H), 6.00 (dd, J=5.8, 3.0 Hz, 1H), 5.70 (dq, J=15.5, 1.8 Hz, 1H), 3.23 (dd, J=3.1, 1.6 Hz, 1H), 2.81 (s, 3H), 1.83 (dd, J=7.0, 1.8 Hz, 3H), 1.71 (s, 1H), 1.61-1.43 (m, 4H).

### Example 24: 2-Methylbicyclo[2.2.1]Hept-5-En-2-Yl (E)-2-Methylbut-2-Enoate

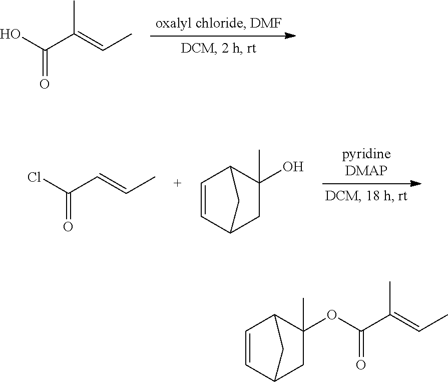

Tiglic acid (1.3 mL, 1.5 Eq, 12.0 mmol) was dissolved in DCM (15 mL) and oxalyl chloride (2.2 mL, 3.2 Eq, 26 mmol) and 2 drops of DMF were added. The reaction mixture was stirred at room temperature for 2h. After full conversion of the acid was achieved, the reaction mixture was stopped and concentrated under reduced pressure affording (E)-2-methylbut-2-enoyl chloride as a brown residue. The crude residue was used as such in the next step. To a stirring solution of bicyclo[2.2.1]heptan-2-ol (1.0 g, 1 Eq, 8.1 mmol) in DCM (10.0 mL) was added (E)-2-methylbut-2-enoyl chloride (1.5 Eq, 12.0 mmol), pyridine (2.0 mL, 3 Eq, 24 mmol) and DMAP (0.20 g, 0.2 Eq, 1.6 mmol). The reaction mixture was stirred for 18 h at room temperature. After reaction completion, the mixture was diluted with water and filtered off over a phase separator. The filtrate was first concentrated under reduced pressure and then purified by flash column chromatography (SiO2, 0-20% MTBE in heptane), affording 2-methylbicyclo[2.2.1]hept-5-en-2-yl (E)-2-methylbut-2-enoate (159 mg, 0.77 mmol, 10% yield) as a colorless oil. GCMS: Method 8, 2.59 min, 206.1, calcd. 206.3; LCMS: Method 2, 2.05 min, M+H=207.1; calcd. 207.285; ¹H NMR (400 MHz, CDCl₃) δ 6.66 (dtt, J=9.0, 5.5, 2.0 Hz, 1H), 6.23 (dd, J=5.7, 3.0 Hz, 1H), 6.01 (dd, J=5.7, 3.0 Hz, 1H), 3.21 (h, J=1.8 Hz, 1H), 2.82 (tt, J=3.4, 1.7 Hz, 1H), 1.86-1.81 (m, 1H), 1.74 (dd, J=5.4, 1.5 Hz, 6H), 1.72 (s, 3H), 1.60-1.56 (m, 1H), 1.54-1.50 (m, 1H), 1.47 (dt, J=8.8, 1.6 Hz, 1H)

### Example 25: 5-Methoxy-5-Methylbicyclo[2.2.1]Hept-2-Ene

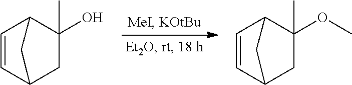

To 2-methylbicyclo[2.2.1]hept-5-en-2-ol (1.20 g, 1 Eq, 9.66 mmol) in diethyl ether (40 mL) were added potassium tert-butoxide (2.17 g, 2 Eq, 19.3 mmol) and iodomethane (0.90 mL, 1.5 Eq, 14.5 mmol). The resulting mixture was stirred at room temperature overnight. After reaction completion, the mixture was diluted with water and diethyl ether and layers were separated. The aqueous layer was extracted twice more with diethyl ether and the combined organics were washed with brine, dried over Na₂SO₄, filtered and concentrated under reduced pressure. The residue was purified by flash column chromatography (SiO₂, 0-25% diethyl ether in pentane), affording 5-methoxy-5-methylbicyclo[2.2.1]hept-2-ene (224 mg, 1.62 mmol, 17% yield) as a colorless oil. GCMS: Method 8, 1.98 min, 138.1, calcd. 138.2; ¹H NMR (400 MHz, CDCl₃) δ 6.27 (dd, J=5.7, 3.0 Hz, 1H), 6.11 (dd, J=5.8, 2.9 Hz, 1H), 3.16 (s, 3H), 2.79 (ddt, J=9.0, 3.2, 1.8 Hz, 2H), 1.64-1.58 (m, 1H), 1.53 (ddt, J=8.8, 3.5, 1.9 Hz, 1H), 1.47 (s, 4H), 1.28 (dd, J=12.0, 3.4 Hz, 1H).

### Example 26: 5-Ethoxy-5-Methylbicyclo[2.2.1]Hept-2-Ene

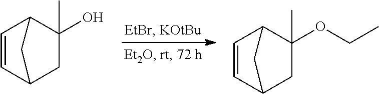

To 2-methylbicyclo[2.2.1]hept-5-en-2-ol (1.20 g, 1 Eq, 9.66 mmol) in diethyl ether (40 mL) were added potassium tert-butoxide (4.34 g, 4 Eq, 38.6 mmol) and ethyl bromide (2.16 mL, 3.0 Eq, 29 mmol). The resulting mixture was stirred at room temperature for 72h. After reaction completion, the mixture was diluted with water and diethyl ether and layers were separated. The aqueous layer was extracted twice more with diethyl ether and the combined organics were washed with brine, dried over Na₂SO₄, filtered and concentrated under reduced pressure. The residue was purified by flash column chromatography (SiO2, 0-30% diethyl ether in pentane), affording 5-ethoxy-5-methylbicyclo[2.2.1]hept-2-ene (311.6 mg, 2.05 mmol, 21% yield) as a colorless oil. GCMS: Method 8, 1.98 min, 138.1, calcd. 138.2; LCMS: Method 2, 1.56 min, M+H=128.8 [fragment mass]; calcd. 153.237; ¹H NMR (400 MHz, CDCl₃) δ 6.26 (tt, J=6.0, 2.6 Hz, 1H), 6.11-6.03 (m, 1H), 3.51-3.37 (m, 1H), 3.31 (tdd, J=9.0, 4.5, 2.0 Hz, 1H), 2.79 (d, J=5.8 Hz, 2H), 1.62 (dt, J=12.3, 3.4 Hz, 1H), 1.53-1.42 (m, 5H), 1.29 (dq, J=11.8, 3.0 Hz, 1H), 1.10 (dddd, J=8.6, 6.8, 3.9, 1.8 Hz, 3H).

### Example 27: 5-Methoxy-5-Vinylbicyclo[2.2.1]Hept-2-Ene

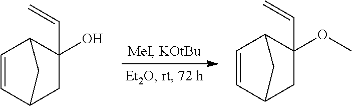

To 2-vinylbicyclo[2.2.1]hept-5-en-2-ol (1.00 g, 1 Eq, 7.34 mmol) in diethyl ether (40 mL) were added potassium tert-butoxide (1.65 g, 2 Eq, 14.7 mmol) and iodomethane (1.56 g, 0.70 mL, 1.5 Eq, 11.0 mmol) and the mixture was stirred at room temperature for 72h. Upon potassium tert-butoxide addition the mixture turned purple. After reaction completion, the mixture was diluted with water and diethyl ether and layers were separated. The aqueous layer was extracted twice more with diethyl ether and the combined organics were washed with brine, dried over Na₂SO₄, filtered and concentrated under reduced pressure. The residue was purified by flash column chromatography (SiO2, 0-30% diethyl ether in pentane), affording 5-methoxy-5-vinylbicyclo[2.2.1]hept-2-ene (761 mg, 5.06 mmol, 69% yield) as colorless oil. GCMS: Method 8, 2.10 min, 150.1, calcd. 150.2; LCMS: Method 2, 1.53 min, M+H=118.9 [fragment mass]; calcd. 151.221. ¹H NMR (400 MHz, CDCl₃) δ 6.36 (dt, J=5.5, 2.7 Hz, 1H), 6.13 (dt, J=5.6, 2.6 Hz, 1H), 6.07-5.96 (m, 1H), 5.31-5.20 (m, 2H), 3.15-3.07 (m, 3H), 2.97 (s, 1H), 2.84 (s, 1H), 1.93-1.83 (m, 1H), 1.55-1.47 (m, 2H), 1.29 (d, J=11.7 Hz, 1H).

### Example 28: 5-Allyl-5-Methoxybicyclo[2.2.1]Hept-2-Ene

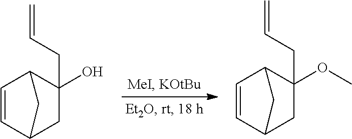

To 2-allylbicyclo[2.2.1]hept-5-en-2-ol (1.00 g, 1 Eq, 6.66 mmol) in Diethyl ether (40 mL) were added potassium tert-butoxide (1.49 g, 2 Eq, 13.3 mmol) and iodomethane (0.62 mL, 1.5 Eq, 9.99 mmol) and the mixture was stirred at room temperature for 18h. After reaction completion, the mixture was diluted with water and diethyl ether and layers were separated. The aqueous layer was extracted twice more with diethyl ether and the combined organics were washed with brine, dried over Na₂SO₄, filtered and concentrated under reduced pressure. The residue was purified by flash column chromatography (SiO₂, 0-30% diethyl ether in pentane), affording 5-allyl-5-methoxybicyclo[2.2.1]hept-2-ene (765 mg, 4.66 mmol, 70% yield) as a colorless oil. GCMS: Method 8, 2.24 min, 164.1, calcd. 164.2; ¹H NMR (400 MHz, CDCl₃) δ 6.29 (dd, J=5.7, 3.0 Hz, 1H), 6.09 (dd, J=5.7, 3.0 Hz, 1H), 5.92 (ddt, J=17.2, 10.3, 6.9 Hz, 1H), 5.15-5.11 (m, 1H), 5.11-5.08 (m, 1H), 3.15 (s, 3H), 2.83 (ddq, J=21.0, 3.6, 1.7 Hz, 2H), 2.71 (dd, J=15.0, 6.7 Hz, 1H), 2.41 (ddt, J=15.0, 7.1, 1.4 Hz, 1H), 1.69 (dd, J=12.2, 3.7 Hz, 1H), 1.57-1.51 (m, 1H), 1.50-1.45 (m, 1H), 1.17 (dd, J=12.1, 3.3 Hz, 1H).

### Example 29: Bicyclo[2.2.1]Heptan-2-Yl Isobutyrate

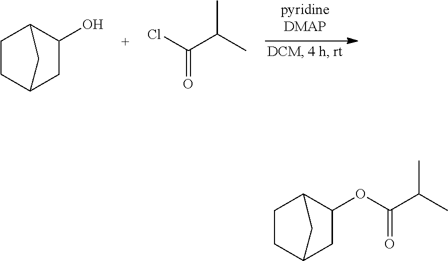

To a stirring solution of bicyclo[2.2.1]heptan-2-ol (1.0 g, 1 Eq, 8.9 mmol) in DCM (10.0 mL) was added isobutyryl chloride (1.4 mL, 1.5 Eq, 13.0 mmol), pyridine (2.1 g, 2.2 mL, 3 Eq, 27 mmol) and DMAP (0.22 g, 0.2 Eq, 1.8 mmol). The reaction mixture was stirred for 4 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-20% MTBE in heptane), affording bicyclo[2.2.1]heptan-2-yl isobutyrate (720g, 3.95 mmol, 44% yield) as a colorless oil (95:5 mixture of isomers). GCMS: Method 8, 2.37 min, 182.1, calcd. 182.2; ¹H NMR (400 MHz, CDCl₃) δ 4.92 (dtd, J=9.7, 4.0, 1.6 Hz, 1H), 2.54 (dq, J=14.0, 7.0 Hz, 1H), 2.47 (td, J=4.2, 1.7 Hz, 1H), 2.23-2.20 (m, 1H), 1.99 (dddd, J=13.3, 10.3, 4.7, 3.1 Hz, 1H), 1.77 (ddt, J=13.4, 8.4, 2.9 Hz, 1H), 1.63-1.52 (m, 1H), 1.38 (dddd, J=11.3, 9.0, 4.2, 1.8 Hz, 2H), 1.31 (ddt, J=9.9, 3.3, 1.9 Hz, 2H), 1.17 (dd, J=7.0, 2.3 Hz, 6H), 0.96 (dt, J=13.3, 3.5 Hz, 1H).

### Example 30: Bicyclo[2.2.1]Heptan-2-Yl Benzoate

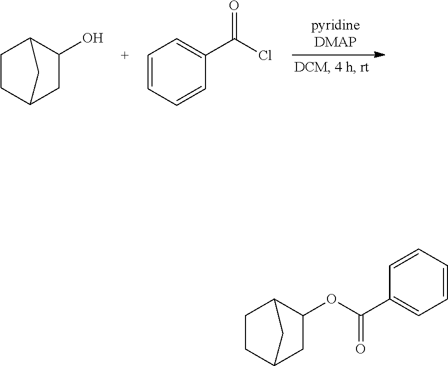 [SMILES: OC1CC2CCC1C2 ; O=C(Cl)c1ccccc1 ; O=C(OC1CC2CCC1C2)c1ccccc1]

To a stirring solution of bicyclo[2.2.1]heptan-2-ol (1.0 g, 1 Eq, 8.9 mmol) in DCM (10.0 mL) was added benzoyl chloride (1.5 mL, 1.5 Eq, 13.0 mmol), pyridine (2.2 mL, 3 Eq, 27 mmol) and DMAP (0.22 g, 0.2 Eq, 1.8 mmol). The reaction mixture was stirred for 4 h at room temperature. After reaction completion, the mixture was then diluted with DCM and washed thrice with water. The combined organic layers were washed with brine, dried over Na₂SO₄, filtered and concentrated under reduced pressure. The crude was then purified by flash column chromatography (SiO₂, 0-20% MTBE in heptane), affording bicyclo[2.2.1]heptan-2-yl benzoate (1.3 g, 6.0 mmol, 67% yield) as a colorless oil (95:5 mixture of isomers). GCMS: Method 8, 3.04 min, 216.1, calcd. 216.3; LCMS: Method 5, 1.99 min, M+H=217.2; calcd. 217.28; ¹H NMR (400 MHz, CDCl₃) δ 8.07-8.03 (m, 2H), 7.60-7.50 (m, 1H), 7.44 (dd, J=8.4, 6.9 Hz, 2H), 5.19 (dtd, J=9.8, 4.0, 1.5 Hz, 1H), 2.65-2.58 (m, 1H), 2.28 (d, J=4.6 Hz, 1H), 2.12 (dddd, J=13.4, 10.4, 4.7, 3.1 Hz, 1H), 1.92 (ddt, J=13.2, 8.3, 2.8 Hz, 1H), 1.70-1.61 (m, 1H), 1.46 (ddt, J=9.4, 6.0, 1.8 Hz, 2H), 1.38 (ddtt, J=9.0, 7.0, 3.6, 1.8 Hz, 2H), 1.15 (dt, J=13.4, 3.5 Hz, 1H).

### Example 31: Bicyclo[2.2.1]Heptan-2-Yl Pivalate

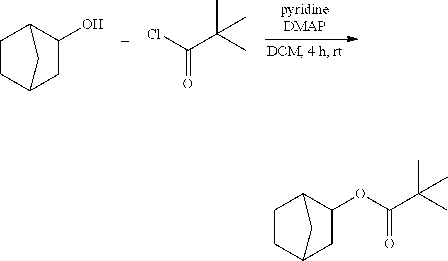

To a stirring solution of bicyclo[2.2.1]heptan-2-ol (1.0 g, 1 Eq, 8.9 mmol) in DCM (10.0 mL) was added trimethylacetyl chloride (1.6 mL, 1.5 Eq, 13.0 mmol), pyridine (2.2 mL, 3 Eq, 27 mmol) and DMAP (0.22 g, 0.2 Eq, 1.8 mmol). The reaction mixture was stirred for 4 h at room temperature. After reaction completion, the mixture was then diluted with DCM and washed thrice with water. The combined organic layers were washed with brine, dried over Na₂SO₄, filtered and concentrated under reduced pressure. The crude was then purified by flash column chromatography (SiO₂, 0-20% MTBE in heptane), affording bicyclo[2.2.1]heptan-2-yl pivalate (482 mg, 2.45 mmol, 28% yield) as a colorless oil (95:5 mixture of isomers). GCMS: Method 8, 2.39 min, 139.0 [fragment mass], calcd. 196.3; LCMS: Method 5, 2.02 min, M+H=136.1 [fragment mass]; calcd. 197.29; ¹H NMR (400 MHz, CDCl₃) δ 4.90 (dddd, J=9.7, 4.7, 3.4, 1.5 Hz, 1H), 2.48 (dq, J=4.8, 2.1 Hz, 1H), 2.22 (t, J=4.6 Hz, 1H), 1.99 (dddd, J=13.3, 10.3, 4.6, 3.0 Hz, 1H), 1.77 (dddd, J=13.8, 8.8, 4.8, 2.4 Hz, 1H), 1.62-1.54 (m, 1H), 1.38 (dddd, J=10.3, 8.8, 3.9, 1.7 Hz, 2H), 1.33-1.25 (m, 2H), 1.20 (s, 9H), 0.94 (dt, J=13.3, 3.5 Hz, 1H).

### Example 32: Bicyclo[2.2.1]Heptan-2-Yl Butyrate

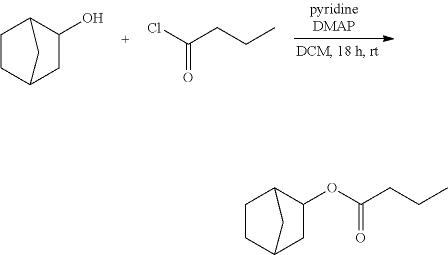

To a stirring solution of bicyclo[2.2.1]heptan-2-ol (1.0 g, 1 Eq, 8.9 mmol) in DCM (10.0 mL) was added n-butyryl chloride (1.4 mL, 1.5 Eq, 13.0 mmol), pyridine (2.2 mL, 3 Eq, 27 mmol) and DMAP (0.22 g, 0.2 Eq, 1.8 mmol). The reaction mixture was stirred for 18 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-20% MTBE in heptane), affording bicyclo[2.2.1]heptan-2-yl butyrate (925 mg, 5.08 mmol, 57% yield) as a colorless oil (95:5 mixture of isomers). GCMS: Method 8, 2.39 min, 154.1 [fragment mass], calcd. 182.2; ¹H NMR (400 MHz, CDCl₃) δ 4.94 (dtd, J=9.8, 4.0, 1.5 Hz, 1H), 2.47 (dq, J=4.5, 2.2 Hz, 1H), 2.28 (t, J=7.4 Hz, 2H), 2.22 (td, J=5.2, 2.9 Hz, 1H), 1.99 (dddd, J=13.3, 10.3, 4.6, 3.0 Hz, 1H), 1.79-1.71 (m, 1H), 1.66 (q, J=7.4 Hz, 2H), 1.56 (dtd, J=12.0, 4.4, 3.0 Hz, 1H), 1.38 (dddd, J=8.4, 6.1, 4.1, 1.8 Hz, 2H), 1.34-1.26 (m, 2H), 1.01-0.93 (m, 4H).

### Example 33: Bicyclo[2.2.1]Heptan-2-Yl (E)-But-2-Enoate

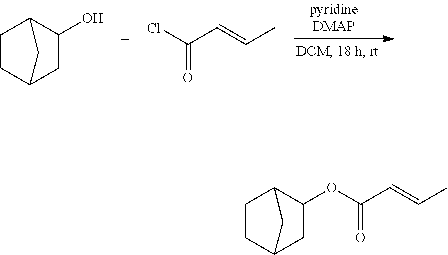

To a stirring solution of bicyclo[2.2.1]heptan-2-ol (1.0 g, 1 Eq, 8.9 mmol) in DCM (10.0 mL) was added (E)-crotonoyl chloride (1.3 mL, 1.5 Eq, 13.0 mmol), pyridine (2.2 mL, 3 Eq, 27 mmol) and DMAP (0.22 g, 0.2 Eq, 1.8 mmol). The reaction mixture was stirred for 18 h at room temperature. After reaction completion, the mixture was diluted with water and filtered off over a phase separator. The filtrate was first concentrated and reduced pressure and then purified by flash column chromatography (SiO₂, 0-20% MTBE in heptane), affording bicyclo[2.2.1]heptan-2-yl (E)-but-2-enoate (385 mg, 2.14 mmol, 24% yield) as a colorless oil (94:6 mixture of isomers). GCMS: Method 8, 2.55 min, 180.1, calcd. 180.2; ¹H NMR (400 MHz, CDCl₃) δ 6.96 (dq, J=15.5, 6.9 Hz, 1H), 5.85 (dq, J=15.6, 1.7 Hz, 1H), 4.99 (dtd, J=9.9, 4.0, 1.5 Hz, 1H), 2.50 (dq, J=4.5, 1.9 Hz, 1H), 2.22 (t, J=4.5 Hz, 1H), 2.01 (dddd, J=13.3, 10.4, 4.7, 3.1 Hz, 1H), 1.87 (dd, J=5.2, 1.6 Hz, 3H), 1.82-1.75 (m, 1H), 1.63-1.55 (m, 2H), 1.39 (ddq, J=11.5, 7.1, 2.4 Hz, 2H), 1.32 (ddt, J=8.4, 3.7, 1.6 Hz, 2H), 1.02 (dt, J=13.3, 3.5 Hz, 1H).

### Example 34: Bicyclo[2.2.1]Heptan-2-Yl (E)-2-Methylbut-2-Enoate

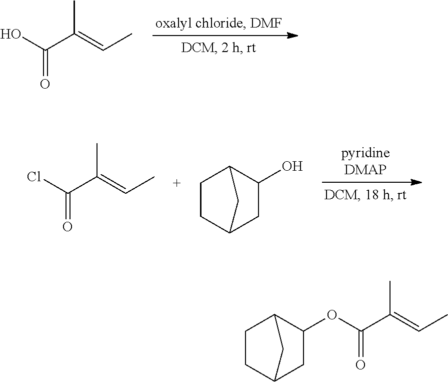

Tiglic acid (1.4 mL, 1.5 Eq, 13.0 mmol) was dissolved in DCM (15 mL) and oxalyl chloride (1.8 mL, 2.35 Eq, 20.7 mmol) and 1 drop of DMF were added. The reaction mixture was stirred at room temperature for 2h. After full conversion of the acid was achieved, the reaction mixture was stopped and concentrated under reduced pressure affording (E)-2-methylbut-2-enoyl chloride (13.0 mmol) as a brown residue. The crude residue was used as such in the next step. To a stirring solution of bicyclo[2.2.1]heptan-2-ol (1.0 g, 1 Eq, 8.9 mmol) in DCM (10.0 mL) was added (E)-2-methylbut-2-enoyl chloride (1.5 Eq, 13.0 mmol), pyridine (2.1 g, 2.2 mL, 3 Eq, 27 mmol) and DMAP (0.22 g, 0.2 Eq, 1.8 mmol). The reaction mixture was stirred for 18 h at room temperature. After reaction completion, the mixture was diluted with water and filtered off over a phase separator. The filtrate was first concentrated under reduced pressure and then purified by flash column chromatography (SiO₂, 0-20% MTBE in heptane), affording bicyclo[2.2.1]heptan-2-yl (E)-2-methylbut-2-enoate (700 mg, 3.60 mmol, 40% yield) as a colorless oil (95:5 mixture of isomers). GCMS: Method 8, 2.64 min, 194.1, calcd. 194.3; LCMS: Method 2, 2.09 min, M+H=195.1; calcd. 195.274; ¹H NMR (400 MHz, CDCl₃) δ 6.85 (dddd, J=8.6, 7.0, 5.5, 1.6 Hz, 1H), 5.04-4.94 (m, 1H), 2.54-2.47 (m, 1H), 2.26-2.19 (m, 1H), 2.03 (tdd, J=10.2, 4.7, 2.3 Hz, 1H), 1.83 (t, J=1.4 Hz, 3H), 1.80-1.78 (m, 4H), 1.63-1.53 (m, 1H), 1.40 (dddd, J=10.2, 8.5, 3.9, 1.7 Hz, 2H), 1.35-1.28 (m, 2H), 1.02 (dt, J=13.3, 3.5 Hz, 1H).

### Example 35: Bicyclo[2.2.1]Heptan-2-Yl (E)-2-Methylbut-2-Enoate

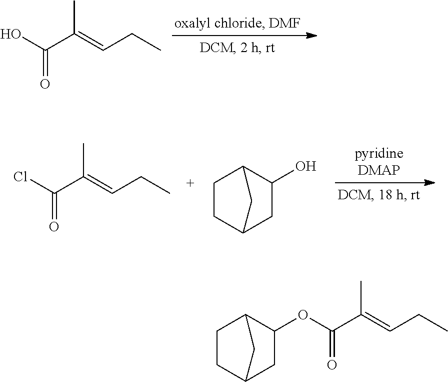

trans-2-Methyl-2-pentenoicacid (1.6 mL, 1.5 Eq, 13.0 mmol) was dissolved in DCM (15 mL) and oxalyl chloride (1.8 mL, 2.35 Eq, 20.7 mmol) and 1 drop of DMF were added. The reaction mixture was stirred at room temperature for 2h. After full conversion of the acid was achieved, the reaction mixture was stopped and concentrated under reduced pressure affording (E)-2-methylpent-2-enoyl chloride as a brown residue (13.0 mmol). The crude residue was used as such in the next step. To a stirring solution of bicyclo[2.2.1]heptan-2-ol (1.0 g, 1 Eq, 8.9 mmol) in DCM (10.0 mL) was added (E)-2-methylpent-2-enoyl chloride (1.5 Eq, 13.0 mmol), pyridine (2.1 g, 2.2 mL, 3 Eq, 27 mmol) and DMAP (0.22 g, 0.2 Eq, 1.8 mmol). The reaction mixture was stirred for 18 h at room temperature. After reaction completion, the mixture was diluted with water and filtered off over a phase separator. The filtrate was first concentrated under reduced pressure and then purified by flash column chromatography (SiO₂, 0-20% MTBE in heptane), affording bicyclo[2.2.1]heptan-2-yl (E)-2-methylbut-2-enoate (870 mg, 4.18 mmol, 47% yield) as a colorless oil (95:5 mixture of isomers). GCMS: Method 8, 2.76 min, 208.1, calcd. 208.3; ¹H NMR (400 MHz, CDCl₃) δ 6.74 (tq, J=7.4, 1.5 Hz, 1H), 4.99 (dtd, J=9.8, 4.0, 1.5 Hz, 1H), 2.50 (td, J=4.1, 1.9 Hz, 1H), 2.25-2.14 (m, 3H), 2.03 (dddd, J=13.3, 10.3, 4.6, 3.0 Hz, 1H), 1.83 (d, J=1.3 Hz, 3H), 1.82-1.76 (m, 1H), 1.64-1.54 (m, 1H), 1.40 (ddt, J=8.3, 7.0, 2.2 Hz, 2H), 1.33 (ddtd, J=11.5, 7.8, 3.8, 2.0 Hz, 2H), 1.08-0.99 (m, 4H).

### Example 36: 2-Methoxybicyclo[2.2.1]Heptane

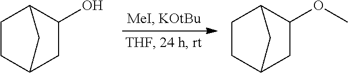

To a stirring solution of bicyclo[2.2.1]heptan-2-ol (900 mg, 1 Eq, 8.02 mmol) in THF (15 mL) was added potassium tert-butoxide (2.70 g, 3 Eq, 24.1 mmol) and iodomethane (1.5 mL, 3 Eq, 24.0 mmol). The reaction mixture was stirred at room temperature for 24h. The reacting mixture was stopped and concentrated under reduced pressure. The residue was diluted with water and DCM and the organics were extracted thrice with DCM. The combined organic layers were dried over Na₂SO₄, filtered and evaporated. The residue was purified by flash column chromatography (SiO₂, 0-20% MTBE in heptane), affording 2-methoxybicyclo[2.2.1]heptane (323 mg, 2.56 mmol, 32% yield) as a colorless oil (95:5 mixture of isomers). GCMS: Method 8, 1.89 min, 126.1, calcd. 126.2; ¹H NMR (400 MHz, CDCl₃) δ 3.70 (dtd, J=9.6, 3.9, 1.4 Hz, 1H), 3.26 (s, 3H), 2.40 (q, J=3.2 Hz, 1H), 2.16 (t, J=4.7 Hz, 1H), 1.86 (dddd, J=13.0, 10.1, 4.8, 3.0 Hz, 1H), 1.80-1.71 (m, 1H), 1.52 (dtt, J=10.8, 5.3, 2.6 Hz, 1H), 1.35-1.25 (m, 4H), 0.92 (dt, J=12.8, 3.2 Hz, 1H).

### Example 37: 2-Ethoxybicyclo[2.2.1]Heptane

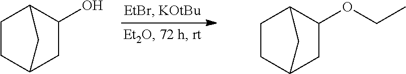

To a stirring solution of bicyclo[2.2.1]heptan-2-ol (1.00 g, 1 Eq, 8.92 mmol) in diethyl ether (30 mL) was added potassium tert-butoxide (2.00 g, 2 Eq, 17.8 mmol) and ethyl bromide (998 μL, 1.5 Eq, 13.4 mmol). The reaction mixture was stirred at room temperature for 72h. The reacting mixture was then diluted with water and diethyl ether and layers were separated. The aqueous layer was extracted twice more with diethyl ether. The combined organic layers were washed with brine, dried over Na₂SO₄, filtered and evaporated under reduced pressure. The residue was purified by flash column chromatography (SiO₂, 0-25% diethyl ether in pentane), affording 2-ethoxybicyclo[2.2.1]heptane (138.3 mg, 0.98 mmol, 11% yield) as colorless oil (75:25 mixture of isomers). GCMS: Method 8, 2.00 min, 140.1, calcd. 140.2; Major: ¹H NMR (400 MHz, CDCl₃) δ 3.79 (dtd, J=9.9, 3.9, 1.5 Hz, 1H), 3.48-3.28 (m, 2H), 2.38 (td, J=4.2, 1.9 Hz, 1H), 2.15 (s, 1H,), 1.90-1.74 (m, 2H), 1.60-1.47 (m, 2H), 1.35-1.26 (m, 3H), 1.19 (t, J=7.0, 3H), 0.93 (dt, J=12.8, 3.4 Hz, 1H). Minor: ¹H NMR (400 MHz, CDCl₃) δ 3.48-3.28 (m, 3H), 2.30 (d, J=4.6 Hz, 1H), 2.24-2.20 (m, 1H), 1.60-1.47 (m, 2H), 1.41 (dddd, J=22.8, 11.4, 4.4, 2.4 Hz, 2H), 1.35-1.26 (m, 3H), 1.17 (t, J=6.8 Hz, 3H), 1.14-0.96 (m, 1H).

### Example 38: 2-(Allyloxy)Bicyclo[2.2.1]Heptane

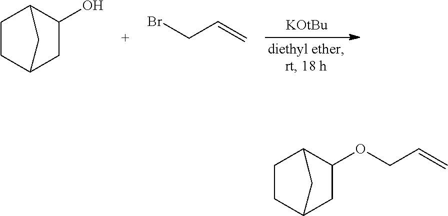

To bicyclo[2.2.1]heptan-2-ol (1.00 g, 1 Eq, 8.92 mmol) in diethyl ether (20 mL) were added potassium tert-butoxide (2.00 g, 2 Eq, 17.8 mmol) and allyl bromide (1.16 mL, 1.5 Eq, 13.4 mmol). The thick orange slurry formed was stirred at room temperature for 18h. After reaction completion, the mixture was diluted with diethyl ether and water and layers were separated. The aqueous layer was extracted thrice more with diethyl ether. The combined organics were washed once with brine, dried over Na₂SO₄, filtered and evaporated under reduced pressure. The crude was purified by flash column chromatography (SiO₂, 0-25% MTBE in pentane), affording 2-(allyloxy)bicyclo[2.2.1]heptane (569.10 mg, 3.74 mmol, 42% yield) as a colorless oil (90:10 mixture of isomers). GCMS: Method 8, 2.16 min, 152.1, calcd. 152.2; ¹H NMR (400 MHz, CDCl₃) δ 5.94 (ddt, J=17.3, 10.8, 5.6 Hz, 1H), 5.27 (dq, J=17.2, 1.8 Hz, 1H), 5.15 (dq, J=10.3, 1.5 Hz, 1H), 3.96-3.91 (m, 1H), 3.88-3.83 (m, 2H), 2.39 (dq, J=4.5, 1.9 Hz, 1H), 2.16 (t, J=4.5, 1H), 1.89-1.78 (m, 2H), 1.55-1.47 (m, 1H), 1.31 (qdd, J=10.0, 5.3, 3.1 Hz, 4H), 0.97 (dt, J=12.7, 3.3 Hz, 1H)

### Example 39: 2-((2-Methylallyl)Oxy)Bicyclo[2.2.1]Heptane

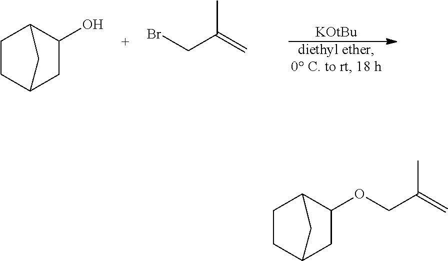

To bicyclo[2.2.1]heptan-2-ol (1.00 g, 1 Eq, 8.92 mmol) in diethyl ether (20 mL) were added potassium tert-butoxide (2.00 g, 2 Eq, 17.8 mmol) and 3-bromo-2-methylprop-1-ene (1.35 mL, 1.5 Eq, 13.4 mmol). The thick orange slurry formed was stirred at room temperature for 18h. After reaction completion, the mixture was diluted with diethyl ether and water and layers were separated. The aqueous layer was extracted thrice more with diethyl ether. The combined organics were washed once with brine, dried over Na₂SO₄, filtered and evaporated under reduced pressure. The crude was purified by flash column chromatography (SiO₂, 0-25% MTBE in pentane), affording 2-((2-methylallyl)oxy)bicyclo[2.2.1]heptane (468.50 mg, 2.8179 mmol, 31.6%) as a colorless oil. GCMS: Method 8, 2.27 min, 151.1 [fragment mass], calcd. 166.2; ¹H NMR (400 MHz, CDCl₃) δ 4.97 (dd, J=2.3, 1.2 Hz, 1H), 4.86 (d, J=2.4 Hz, 1H), 3.88-3.79 (m, 2H), 3.74 (d, J=12.4 Hz, 1H), 2.40-2.36 (m, 1H), 2.16 (t, J=4.8 Hz, 1H), 1.84 (dddd, J=15.6, 8.0, 3.7, 2.1 Hz, 2H), 1.75 (d, J=1.1 Hz, 3H), 1.58-1.48 (m, 2H), 1.36-1.25 (m, 3H), 0.97 (dt, J=12.8, 3.4 Hz, 1H).

### Example 40: 2-((3-Methylbut-2-En-1-Yl)Oxy)Bicyclo[2.2.1]Heptane

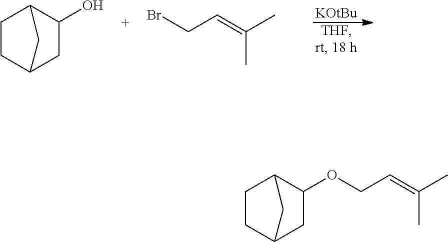

To bicyclo[2.2.1]heptan-2-ol (700 mg, 1 Eq, 6.24 mmol) in diethyl ether (40 mL) was added potassium tert-butoxide (1.40 g, 2 Eq, 12.5 mmol) and 3,3-dimethylallylbromide (1.09 mL, 1.5 Eq, 9.36 mmol). The reaction was stirred at room temperature for 18 h. After reaction completion, the mixture was diluted with water and extracted thrice with diethyl ether, layers were separated, and the organics were dried over Na₂SO₄ and concentrated. The crude mixture was purified by flash column chromatography (SiO₂, 0-25% Et₂O in pentane). Purification was then repeated (SiO₂, 0-30% EtOAc in heptane) to give 2-((3-methylbut-2-en-1-yl)oxy)bicyclo[2.2.1]heptane (341 mg, 1.89 mmol, 30% yield) as a colorless oil. GCMS: Method 8, 2.51 min. 180.1, calcd. 180.2; ¹H NMR (400 MHz, CDCl₃) δ 5.42-5.29 (m, 1H), 3.92 (dd, J=11.5, 6.8 Hz, 1H), 3.85-3.78 (m, 1H), 2.43-2.31 (m, 1H), 2.26-2.11 (m, 1H), 1.89-1.79 (m, 1H), 1.74 (d, J=1.2 Hz, 3H), 1.67 (d, J=1.4 Hz, 3H), 1.60-1.47 (m, 2H), 1.30 (dtdd, J=12.7, 8.2, 4.1, 2.5 Hz, 4H), 0.97 (dt, J=12.7, 3.3 Hz, 1H), 0.91-0.86 (m, 1H).

### Example 41: 2-Methoxy-2-Vinylbicyclo[2.2.1]Heptane

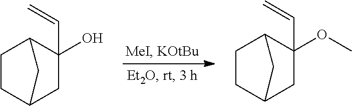

To 2-vinylbicyclo[2.2.1]heptan-2-ol (1.00 g, 1 Eq, 7.24 mmol) in diethyl ether (30 mL) were added KOtBu (1.62 g, 2 Eq, 14.5 mmol) and iodomethane (679 μL, 1.5 Eq, 10.9 mmol). The resulting mixture was stirred at room temperature for 3h. After reaction completion, the mixture was diluted with diethyl ether and water and layers were separated. The aqueous one was extracted twice more with diethyl ether, then the organics were combined, washed one last time with brine and then dried over Na₂SO₄, filtered and evaporated under reduced pressure. The crude was purified by flash column chromatography (SiO₂, 0-25% diethyl ether in pentane), affording 2-methoxy-2-vinylbicyclo[2.2.1]heptane (693 mg, 4.55 mmol, 63% yield) as a colorless oil. GCMS: Method 8, 2.14 min, 152.1, calcd. 152.2; ¹H NMR (400 MHz, CDCl₃) δ 5.77 (dd, J=17.7, 11.0 Hz, 1H), 5.21-5.08 (m, 2H), 3.10 (s, 3H), 2.35 (t, J=5.0 Hz, 1H), 2.21 (t, J=5.0 Hz, 1H), 1.85 (tdd, J=10.9, 5.5, 2.5 Hz, 1H), 1.75 (ddd, J=13.4, 4.9, 2.9 Hz, 1H), 1.60-1.50 (m, 1H), 1.50-1.45 (m, 1H), 1.42-1.29 (m, 2H), 1.27-1.20 (m, 2H).

### Example 42: 2-Ethoxy-2-Vinylbicyclo[2.2.1]Heptane

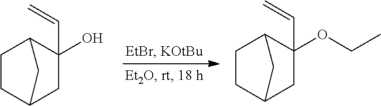

To 2-vinylbicyclo[2.2.1]heptan-2-ol (1.00 g, 1 Eq, 7.24 mmol) in diethyl ether (30 mL) were added potassium tert-butoxide (1.62 g, 2 Eq, 14.5 mmol) and bromoethane (0.81 mL, 1.5 Eq, 10.9 mmol). The resulting mixture was stirred at room temperature for 18h. After reaction completion, the mixture was diluted with diethyl ether and water and layers were separated. The aqueous layer was extracted twice more with diethyl ether, then the organics were combined, washed one last time with brine and then dried over Na₂SO₄, filtered and evaporated under reduced pressure. The crude was purified by flash column chromatography (SiO₂, 0-30% diethyl ether in pentane), affording 2-ethoxy-2-vinylbicyclo[2.2.1]heptane (426.9 mg, 2.568 mmol, 35.5%) as colorless oil. GCMS: Method 8, 2.19 min, 166.1, calcd. 166.2; ¹H NMR (400 MHz, CDCl₃) δ 5.81 (dddd, J=17.9, 11.1, 7.3, 2.8 Hz, 1H), 5.18-5.05 (m, 2H), 3.37-3.14 (m, 2H), 2.36 (s, 1H), 2.20 (s, 1H), 1.95-1.81 (m, 1H), 1.76 (s, 1H), 1.54-1.43 (m, 2H), 1.36 (dt, J=14.8, 7.7 Hz, 2H), 1.25 (dt, J=17.9, 5.5 Hz, 2H), 1.15 (qd, J=7.1, 2.6 Hz, 3H).

### Example 43: 2-Allyl-2-Methoxybicyclo[2.2.1]Heptane

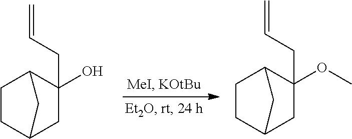

To 2-allylbicyclo[2.2.1]heptan-2-ol (1.00 g, 1 Eq, 6.57 mmol) in diethyl ether (40 mL) were added potassium tert-butoxide (1.47 g, 2 Eq, 13.1 mmol) and iodomethane (0.62 mL, 1.5 Eq, 9.85 mmol). The resulting mixture was stirred at room temperature for 24h. After reaction completion, the mixture was diluted with diethyl ether and water, layers were separated. The aqueous layer was extracted twice more with diethyl ether, then the organics were combined, washed one last time with brine and then dried over Na₂SO₄, filtered and evaporated under reduced pressure. The crude was purified by flash column chromatography (SiO₂, 0-30% diethyl ether in pentane), affording 2-allyl-2-methoxybicyclo[2.2.1]heptane (804.90 mg, 4.84 mmol, 74% yield) as colorless oil. GCMS: Method 8, 2.27 min, 125.1 [fragment mass], calcd. 166.2; ¹H NMR (400 MHz, CDCl₃) δ 5.82 (ddt, J=15.8, 11.7, 6.9 Hz, 1H), 5.11 (t, J=1.3 Hz, 1H), 5.09-5.05 (m, 1H), 3.17 (s, 3H), 2.52 (ddq, J=15.0, 6.6, 1.3 Hz, 1H), 2.22-2.12 (m, 3H), 1.79 (s, 1H), 1.60-1.52 (m, 2H), 1.46 (dt, J=10.1, 2.0 Hz, 1H), 1.34-1.25 (m, 3H), 1.12 (dd, J=12.7, 3.3 Hz, 1H).

### Example 44: 2-Allylbicyclo[2.2.1]Heptan-2-Yl Propionate

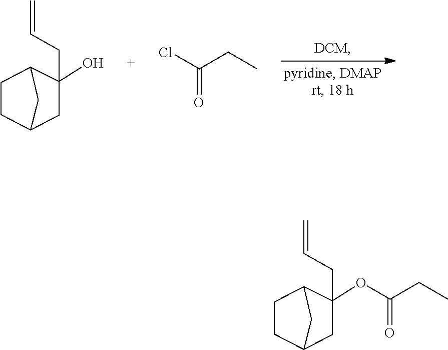

To 2-allylbicyclo[2.2.1]heptan-2-ol (1.00 g, 1 Eq, 6.57 mmol) in DCM (10 mL) was added pyridine (1.59 mL, 3 Eq, 19.7 mmol), DMAP (160 mg, 0.2 Eq, 1.31 mmol) and propionyl chloride (0.86 mL, 1.5 Eq, 9.85 mmol). The reaction was stirred at room temperature for 18h. After reaction completion, the mixture was diluted with DCM and washed thrice with water, layers were separated, and the organics were dried over Na₂SO₄ and concentrated. The crude mixture was purified by flash column chromatography (SiO₂, 0-30% MTBE in heptanes) to give 2-allylbicyclo[2.2.1]heptan-2-yl propionate (336.4 mg, 1.62 mmol, 25% yield) as a colorless oil. GCMS: Method 8, 2.61 min. 167.1 [fragment mass], calcd. 208.1; ¹H NMR (400 MHz, CDCl₃) δ 5.81-5.66 (m, 1H), 5.09-4.98 (m, 2H), 2.88-2.82 (m, 0.5H), 2.82-2.77 (m, 0.5H), 2.71-2.65 (m, 1H), 2.53 (dt, J=7.3, 1.3 Hz, 0.5H), 2.49 (dt, J=7.1, 1.3 Hz, 0.5H), 2.26 (q, J=7.6 Hz, 2H), 2.23-2.17 (m, 1H), 1.76 (dd, J=4.5, 2.8 Hz, 0.5H), 1.73 (dd, J=4.5, 2.8 Hz, 0.5H), 1.62-1.17 (m, 7H), 1.11 (t, J=7.6 Hz, 3H).

### Example 45: 2-Methylbicyclo[2.2.1]Heptan-2-Yl Isobutyrate

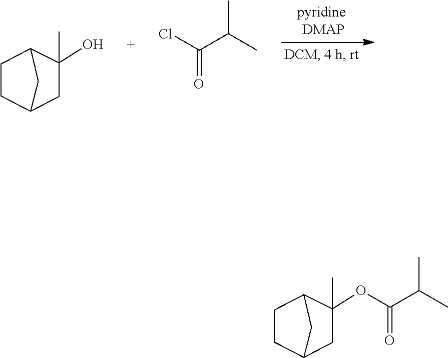

To a stirring solution of 2-methylbicyclo[2.2.1]heptan-2-ol (1.00 g, 1 Eq, 7.92 mmol) in DCM (10 mL) were added pyridine (1.92 mL, 3 Eq, 23.8 mmol), DMAP (97 mg, 0.1 Eq, 0.79 mmol) and isobutyryl chloride (1.25 mL, 1.5 Eq, 11.9 mmol). The reaction mixture was stirred for 4 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-25% MTBE in heptane), affording 2-methylbicyclo[2.2.1]heptan-2-yl isobutyrate (1.07 g, 5.45 mmol, 69% yield) as a colorless oil. GCMS: Method 8, 2.4 min, 196.1, calcd. 196.3; LCMS: Method 5, 1.99 min, M+H=109.1 [fragment mass]; calcd. 197.29; ¹H NMR (400 MHz, CDCl₃) δ 2.57 (dq, J=2.9, 1.4 Hz, 1H), 2.47 (p, J=7.0 Hz, 1H), 2.19 (td, J=4.5, 2.5 Hz, 1H), 1.69-1.59 (m, 2H), 1.57-1.44 (m, 6H), 1.40-1.31 (m, 1H), 1.26 (ddd, J=8.6, 3.5, 1.8 Hz, 1H), 1.23-1.17 (m, 1H), 1.14 (dd, J=7.0, 2.1 Hz, 6H).

### Example 46: 2-Methylbicyclo[2.2.1]Heptan-2-Yl Butyrate

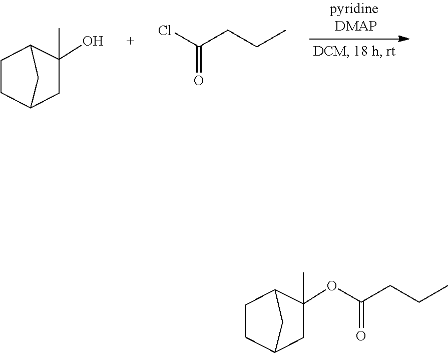

To a stirring solution of 2-methylbicyclo[2.2.1]heptan-2-ol (1.00 g, 1 Eq, 7.92 mmol) in DCM (10 mL) were added pyridine (1.92 mL, 3 Eq, 23.8 mmol), DMAP (97 mg, 0.1 Eq, 0.79 mmol) and n-butyryl chloride (1.24 mL, 1.5 Eq, 11.9 mmol). The reaction mixture was stirred for 4 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-25% MTBE in heptane), affording 2-methylbicyclo[2.2.1]heptan-2-yl butyrate (754 mg, 3.84 mmol, 49% yield) as a colorless oil. GCMS: Method 8, 2.46 min, 196.1, calcd. 196.3; ¹H NMR (400 MHz, CDCl₃) δ 2.57 (dq, J=2.8, 1.3 Hz, 1H), 2.22 (t, J=7.4 Hz, 2H), 2.20-2.17 (m, 1H), 1.68-1.50 (m, 6H), 1.47 (s, 4H), 1.44 (p, J=1.8 Hz, 1H), 1.41-1.31 (m, 1H), 1.28-1.16 (m, 2H), 0.95 (t, J=7.4 Hz, 3H).

### Example 47: 2-Methylbicyclo[2.2.1]Heptan-2-Yl Pivalate

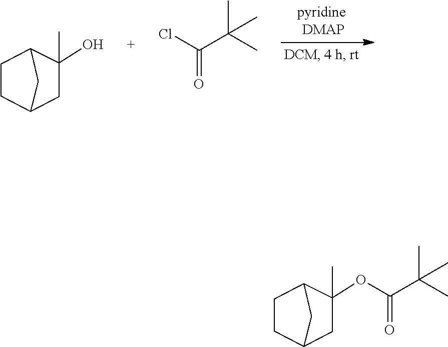

To a stirring solution of 2-methylbicyclo[2.2.1]heptan-2-ol (1.00 g, 1 Eq, 7.92 mmol) in DCM (10 mL) were added pyridine (1.92 mL, 3 Eq, 23.8 mmol), DMAP (97 mg, 0.1 Eq, 0.79 mmol) and pivaloyl chloride (1.46 mL, 1.5 Eq, 11.9 mmol). The reaction mixture was stirred for 4 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-25% MTBE in heptane), affording 2-methylbicyclo[2.2.1]heptan-2-yl pivalate (613 mg, 2.91 mmol, 37% yield) as a colorless oil. GCMS: Method 8, 2.43 min, 153.1 [fragment mass], calcd. 210.3; LCMS: Method 5, 2.13 min, M+H=109.1 [fragment mass]; calcd. 211.317; ¹H NMR (400 MHz, CDCl₃) δ 2.59-2.53 (m, 1H), 2.19 (d, J=4.7 Hz, 1H), 1.70-1.58 (m, 2H), 1.57-1.45 (m, 3H), 1.45 (s, 3H), 1.40-1.31 (m, 1H), 1.29-1.24 (m, 1H), 1.17 (s, 10H).

### Example 48: 2-Methylbicyclo[2.2.1]Heptan-2-Yl Benzoate

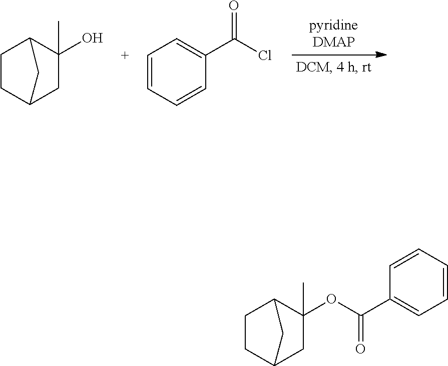

To a stirring solution of 2-methylbicyclo[2.2.1]heptan-2-ol (1.00 g, 1 Eq, 7.92 mmol) in DCM (10 mL) were added pyridine (1.92 mL, 3 Eq, 23.8 mmol), DMAP (97 mg, 0.1 Eq, 0.79 mmol) and benzoyl chloride (1.38 mL, 1.5 Eq, 11.9 mmol). The reaction mixture was stirred for 4 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-25% MTBE in heptane), affording 2-methylbicyclo[2.2.1]heptan-2-yl benzoate (823 mg, 3.58 mmol, 45% yield) as a colorless oil. GCMS: Method 8, 3.05 min, 230.1, calcd. 230.3; LCMS: Method 5, 2.08 min, M+H=109.1 [fragment mass]; calcd. 231.307; ¹H NMR (400 MHz, CDCl₃) δ 8.03-7.99 (m, 2H), 7.56-7.51 (m, 1H), 7.43 (dd, J=8.4, 7.0 Hz, 2H), 2.76-2.69 (m, 1H), 2.29-2.22 (m, 1H), 1.83-1.67 (m, 3H), 1.61 (s, 3H), 1.60-1.51 (m, 2H), 1.44 (dt, J=12.4, 4.4 Hz, 1H), 1.33 (ddt, J=10.2, 3.3, 1.7 Hz, 1H), 1.26 (tdd, J=9.3, 4.8, 2.4 Hz, 1H).

### Example 49: 2-Methylbicyclo[2.2.1]Heptan-2-Yl (E)-But-2-Enoate

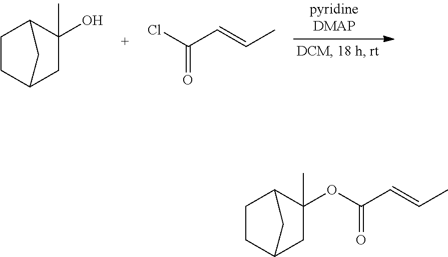

To a stirring solution of 2-methylbicyclo[2.2.1]heptan-2-ol (1.00 g, 0.873 Eq, 7.92 mmol) in DCM (10 mL) were added pyridine (2.20 mL, 3 Eq, 27.2 mmol), DMAP (222 mg, 0.2 Eq, 1.82 mmol) and (E)-but-2-enoyl chloride (1.31 mL, 1.5 Eq, 13.6 mmol). The reaction mixture was stirred for 18 h at room temperature. After reaction completion, the mixture was then diluted with DCM and washed thrice with water. The aqueous layer was back extracted once with DCM and then the combined organic layers were washed with brine, dried over Na₂SO₄, filtered and concentrated under reduced pressure. The crude was then purified by flash column chromatography (SiO₂, 0-25% MTBE in heptane), affording 2-methylbicyclo[2.2.1]heptan-2-yl (E)-but-2-enoate (217.7 mg, 1.12 mmol, 14% yield) as a colorless oil. GCMS: Method 8, 2.57 min, 194.1, calcd. 194.3; LCMS: Method 5, 1.92 min, M+H=195.2; calcd. 195.274. ¹H NMR (400 MHz, CDCl₃) δ 6.89 (dq, J=15.5, 6.9 Hz, 1H), 5.80 (dq, J=15.5, 1.7 Hz, 1H), 2.61 (dq, J=2.8, 1.3 Hz, 1H), 2.19 (td, J=4.4, 2.1 Hz, 1H), 1.86 (dd, J=7.0, 1.7 Hz, 3H), 1.71-1.51 (m, 4H), 1.50 (s, 3H), 1.49-1.44 (m, 1H), 1.41-1.32 (m, 1H), 1.27 (ddt, J=10.2, 3.4, 1.6 Hz, 1H), 1.21 (dddd, J=13.2, 8.8, 5.1, 2.4 Hz, 1H).

### Example 50: 2-Methylbicyclo[2.2.1]Heptan-2-Yl (E)-2-Methylbut-2-Enoate

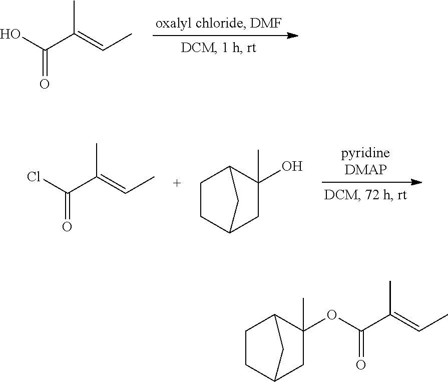

Tiglic acid (3.63 mL, 1 Eq, 35 mmol) was dissolved in DCM (50 mL) and oxalyl chloride (4.6 mL, 1.5 Eq, 52 mmol) and 1 drop of DMF were added. The reaction mixture was stirred at room temperature for 1h. After full conversion of the acid was achieved, the reaction mixture was stopped and concentrated under reduced pressure affording (E)-2-methylbut-2-enoyl chloride (4.1 g, 35 mmol, 99% yield) as a brown residue. Part of the crude residue was used as such in the next step. 2-methylbicyclo[2.2.1]heptan-2-ol (1.00 g, 1 Eq, 7.92 mmol) in DCM (20 mL) was added (E)-2-methylbut-2-enoyl chloride (1.4 g, 1.5 Eq, 11.9 mmol), pyridine (1.92 mL, 3 Eq, 23.8 mmol) and DMAP (194 mg, 0.2 Eq, 1.58 mmol). The reaction mixture was stirred for 72 h at room temperature. After reaction completion, the mixture was diluted with water and filtered off over a phase separator. The filtrate was first concentrated and reduced pressure and then purified by flash column chromatography (SiO₂, 0-20% MTBE in heptane), affording 2-methylbicyclo[2.2.1]heptan-2-yl (E)-2-methylbut-2-enoate (412.90 mg, 1.9822 mmol, 25% yield) as a colorless oil. GCMS: Method 8, 2. min, 208.0, calcd. 208.3; LCMS: Method 2, 2.05 min, M+H=209.2; calcd. 209.301; ¹H NMR (400 MHz, CDCl₃) δ 6.77 (qq, J=7.0, 1.5 Hz, 1H), 2.61 (d, J=4.0 Hz, 1H), 2.24-2.17 (m, 1H), 1.80 (q, J=1.3 Hz, 3H), 1.78 (dt, J=7.0, 1.2 Hz, 3H), 1.72-1.53 (m, 4H), 1.50 (s, 3H), 1.50-1.45 (m, 1H), 1.41-1.32 (m, 1H), 1.28 (ddt, J=9.9, 2.9, 1.5 Hz, 1H), 1.21 (ddt, J=11.4, 7.2, 2.2 Hz, 1H).

### Example 51: 2-Methyl-2-((2-Methylallyl)Oxy)Bicyclo[2.2.1]Heptane

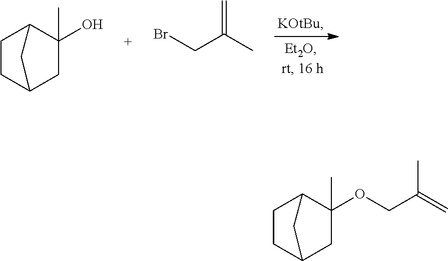

To 2-methylbicyclo[2.2.1]heptan-2-ol (1.00 g, 1 Eq, 7.92 mmol) in diethyl ether (30 mL) was added potassium tert-butoxide (1.78 g, 2 Eq, 15.8 mmol) and 3-Bromoisobutylene (1.20 mL, 1.5 Eq, 11.9 mmol). Stirring at room temperature for 18 h. Due to incomplete conversion, additional potassium tert-butoxide (1.78 g, 2 Eq, 15.8 mmol) and 3-Bromoisobutylene (1.20 mL, 1.5 Eq, 11.9 mmol) were added. Stirring was continued for additional 72 h. After reaction completion, the solution was diluted with water and extracted twice with diethyl ether, layers were separated, and the organics were combined and further washed with brine, then dried over Na₂SO₄, filtered and concentrated. The crude mixture was purified by flash column chromatography (SiO₂, 0-40% MTBE in heptane) to give 2-methyl-2- ((2-methylallyl)oxy)bicyclo[2.2.1]heptane (434 mg, 2.41 mmol, 30% yield) as a colorless oil. GCMS: Method 8, 2.33 min. 165.1 [fragment mass], calcd. 180.1; ¹H NMR (400 MHz, CDCl₃) δ 5.01 (dd, J=2.5, 1.3 Hz, 1H), 4.83 (dq, J=2.6, 1.4 Hz, 1H), 3.74 (q, J=12.5 Hz, 2H), 2.20-2.14 (m, 2H), 1.89 (tt, J=8.7, 2.5 Hz, 1H), 1.74 (s, 3H), 1.48 (dddd, J=23.0, 12.8, 4.4, 2.9 Hz, 3H), 1.33 (dd, J=12.1, 3.0 Hz, 1H), 1.30-1.23 (m, 6H).

### Example 52: 2-Methoxy-2-Methylbicyclo[2.2.1]Heptane

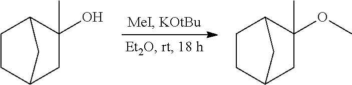

To 2-methylbicyclo[2.2.1]heptan-2-ol (1.00 g, 1 Eq, 7.92 mmol) in diethyl ether (40 mL) were added potassium tert-butoxide (1.78 g, 2 Eq, 15.8 mmol) and iodomethane (0.74 mL, 1.5 Eq, 11.9 mmol). The resulting mixture was stirred at room temperature for 18h. After reaction completion, the mixture was diluted with diethyl ether and water and layers were separated. The aqueous layer was extracted twice more with diethyl ether, then the organics were combined, washed one last time with brine and then dried over Na₂SO₄, filtered and evaporated under reduced pressure. The crude was purified by flash column chromatography (SiO₂, 0-30% diethyl ether in pentane), affording 2-methoxy-2-methylbicyclo[2.2.1]heptane (417 mg, 2.97 mmol, 37% yield) as a colorless oil. GCMS: Method 8, 2.02 min, 140.1, calcd. 140.2; ¹H NMR (400 MHz, CDCl₃) δ 3.17 (s, 3H), 2.20-2.11 (m, 2H), 1.79 (tt, J=10.2, 3.8 Hz, 1H), 1.59-1.43 (m, 3H), 1.33-1.24 (m, 4H), 1.23 (s, 3H).

### Example 53: Spiro[Bicyclo[2.2.1]Heptane-2,2′-Pyran]-6′(3′H)-One

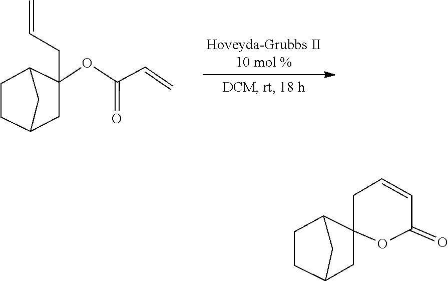

A 2-necked flask equipped with an outlet was dried under vacuum with a heat gun and then let it cool down to room temperature under argon flow. Then, DCM (0.460 L) previously degassed for 30 min, was added to the flask. To 2-allylbicyclo[2.2.1]heptan-2-yl acrylate (560 mg, 1 Eq, 2.71 mmol) in DCM (150 mL) was added dichloro (1,3-dimesityl-2-imidazolidinylidene)(2-isopropoxybenzylidene)ruthenium (170 mg, 0.1 Eq, 271 μmol) and the mixture was stirred at room temperature for 18 h. After reaction completion, the mixture was evaporated under reduced pressure affording a crude oil. The mixture was purified by flash column chromatography (SiO₂, 0-30% MTBE in heptane) to give spiro [bicyclo[2.2.1]heptane-2,2′-pyran]-6′ (3′H)-one (410 mg, 2.30 mmol, 85% yield) as a yellow oil (as a 1:1 mixture of isomers). GCMS: Method 8, 2.99 min. 178.0, calcd. 178.1; ¹H NMR (400 MHz, CDCl₃) δ 6.80 (dt, J=9.7, 4.2 Hz, 1H), 6.04 (dt, J=9.7, 1.9 Hz, 1H), 2.49-2.45 (m, 2H), 2.43-2.40 (m, 1H), 2.26 (t, J=4.6 Hz, 1H), 2.01 (ddt, J=11.7, 8.5, 2.8 Hz, 1H), 1.58 (s, 3H), 1.46-1.33 (m, 4H).

### Example 54: 5′H-Spiro[Bicyclo[2.2.1]Heptane-2,2′-Furan]-5′-One

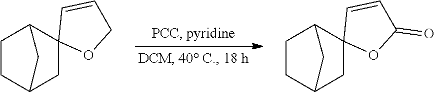 [SMILES: C1=CC2(CC3CCC2C3)OC1 ; O=C1C=CC2(CC3CCC2C3)O1]

To a solution of 5′H-spiro [bicyclo[2.2.1]heptane-2,2′-furan] (450 mg, 1 Eq, 3.00 mmol) in DCM (15.0 mL), pyridine (485 μL, 2.0 Eq, 5.99 mmol) and PCC (1.29 g, 2.0 Eq, 5.99 mmol) were added and the resulting mixture was stirred at 40° C. for 18 h. After reaction completion, the reaction was stopped and allowed to cool down to room temperature. The mixture was then filtered off over a plug of celite and the filter cake was rinsed thrice with DCM. The filtrate was concentrated under reduced pressure. The residue was diluted with more DCM and washed two times with aq. sat. Na₂S₂O₃, then once with water and finally once more with brine. The organics were dried over Na₂SO₄, filtered and evaporated under reduced pressure to afford a crude material. The crude was coated on celite and then purified flash column chromatography (SiO₂, 0-15% MTBE in heptane), affording 5′H-spiro[bicyclo[2.2.1]heptane-2,2′-furan]-5′-one (235 mg, 1.43 mmol, 48% yield) as a white solid (ratio of isomers not determined). GCMS: Method 8, 2.74 min, 164.0, calcd. 164.2.

### Example 55: Dihydro-5′H-Spiro[Bicyclo[2.2.1]Heptane-2,2′-Furan]-5′-One

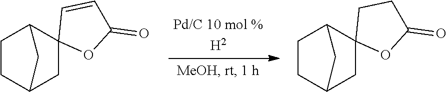 [SMILES: O=C1C=CC2(CC3CCC2C3)O1 ; O=C1CCC2(CC3CCC2C3)O1]

A stirred solution of 5′H-spiro [bicyclo[2.2.1]heptane-2,2′-furan]-5′-one (350 mg, 1 Eq, 2.13 mmol) in MeOH (11 mL) was purged with nitrogen for 5 min. Palladium, 10% on activated carbon, (50% wet with water) (0.454 g, 5% Wt, 0.1 Eq, 0.21 mmol) was added and the solution was purged with nitrogen for another 5 min. The atmosphere was replaced by hydrogen, by submitting the mixture to three cycles of vacuum/hydrogen, and the resulting mixture was stirred at room temperature for 1h. After reaction completion, the atmosphere was purged with nitrogen for a few min. The mixture was then filtered off over a plug of celite and the filter cake was washed thrice with ethanol and evaporated under reduced pressure, affording a crude colorless oil. The oil was filtered again over a silica plug, rinsing three times with MTBE and the filtrate was evaporated, affording dihydro-5′H-spiro [bicyclo[2.2.1]heptane-2,2′-furan]-5′-one (331 mg, 2.0 mmol, 93% yield) as a colorless oil which upon standing turned into a colorless glass solid. GCMS: Method 11, 3.23 min, 166.2, calcd. 166.2. LCMS: Method 4, 2.24 min, M+H=167.0, calcd. 167.199. ¹H NMR (400 MHz, CDCl₃) δ 2.54 (ddd, J=8.9, 6.5, 4.2 Hz, 2H), 2.28 (d, J=4.5 Hz, 1H), 2.24-2.15 (m, 2H), 2.01 (dt, J=12.7, 9.3 Hz, 1H), 1.91 (dtd, J=11.5, 6.3, 2.6 Hz, 1H), 1.74-1.64 (m, 2H), 1.64-1.58 (m, 1H), 1.47-1.35 (m, 4H).

### Example 56: Dihydrospiro[Bicyclo[2.2.1]Heptane-2,2′-Pyran]-6′(3′H)-One

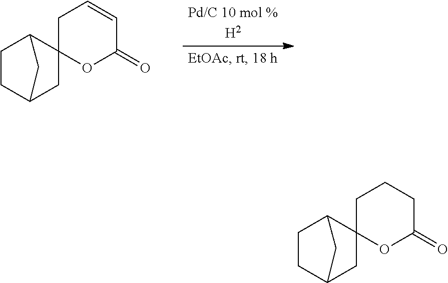 [SMILES: O=C1C=CCC2(CC3CCC2C3)O1 ; O=C1CCCC2(CC3CCC2C3)O1]

A stirred solution of spiro[bicyclo[2.2.1]heptane-2,2′-pyran]-6′(3′H)-one (260 mg, 1 Eq, 1.46 mmol) in EtOAc (20 mL) was purged with nitrogen for 5 min. Palladium, 10% on activated carbon, (310 mg, 5% Wt, 0.1 Eq, 146 μmol) was added and the solution was purged with nitrogen for another 5 min. The atmosphere was replaced by hydrogen, by submitting the mixture to three cycles of vacuum/hydrogen. The mixture was stirred at room temperature for 18h. After reaction completion, the atmosphere was purged with nitrogen for a few min. The mixture was then filtered off over a plug of celite and the filter cake was washed thrice with EtOAc. The filtrate was then concentrated affording a crude residue. The crude mixture was purified by flash column chromatography (SiO₂, 0-100% MTBE in heptanes) to afford spiro dihydrospiro[bicyclo[2.2.1]heptane-2,2′-pyran]-6′(3′H)-one (172.4 mg, 0.95 mmol, 66% yield) as colorless oil. GCMS: Method 8, 2.91 min. 180.1, calcd. 180.1; ¹H NMR (400 MHz, CDCl₃) δ 2.48 (td, J=7.0, 3.9 Hz, 2H), 2.31 (d, J=3.8 Hz, 1H), 2.29-2.24 (m, 1H), 2.12-2.04 (m, 1H), 1.90-1.82 (m, 2H), 1.77 (ddd, J=15.2, 12.0, 6.5 Hz, 2H), 1.71-1.63 (m, 1H), 1.59 (qd, J=8.2, 4.6 Hz, 2H), 1.49-1.44 (m, 2H), 1.43-1.33 (m, 2H).

### Example 57: Odor Profile Determination

Odor profile for selected compounds within the scope of the disclosure is determined using assessments by one Master Perfumer and 4-6 trained sensory panelists. Sensory panelists are trained on our internal taxonomy with reference materials that include 11 grand families, and 62 subfamilies. Panelists are experienced in, but not trained in, an additional several hundred odor descriptors. Panelists are trained on usage of a rating scale, which also includes internal references. Panelists are not advanced to compound evaluation until they have completed and received a passing score on a Final Exam on the taxonomy that was curated by our perfumery team.

A single score is generated for the sensory panel by taking the arithmetic mean of all panelist scores for a given sample, for a given attribute. Alternatively, a single score may be generated by fitting all panelist scores to a model that can correct for differences in the intercept or slope of the latent function through which each panelist produces a rating given a percept, i.e. correction for inter-panelist variation.

The Master Perfumer, possessing a deep knowledge of fragrance ingredients, high level technical expertise, and who is recognized for their experience in creating complex perfumes, reviews, may augment sensory panel scores for improved accuracy.

Test samples are dissolved at a concentration of 10% w/v in ethanol. At time 0 (t=0), new test blotters (White Paper Paddle Shaped Perfumery Blotters, measuring 5×0.5 inches) are dipped into the 10% solution of the text compound. Odor descriptions at time t=0 are captured within 1-2 minutes of wetting the blotter to allow for evaporation of most of the ethanol solvent, permitting a more accurate determination of the test compound's odor. Assessments are made at ambient temperature in a benchtop laboratory setting. The procedure is repeated at 2 hours, 4-6 hours, and at 24 hours.

The results are shown in the table below:

Intensity: 1-10 (max) at t = 0, 10% EtOH solution on blotter paper

| | | | Intensity (1- | Intensity | Intensity |
|---|---|---|---|---|---|
| Ex. | | | 10) at 0 | (1-10) at 4- | (1-10) at |
| No. | Structure | Primary Notes | hours | 6 hours | 24 hours |
| 1 | 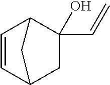 | mineral, earthy, wet, mossy, ozonic, mushroom | 4 | 4 | 3 |
| 2 | 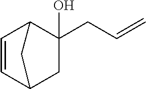 | herbal, woody, lavender, earthy | 6 | 3 | 1 |
| 3 | 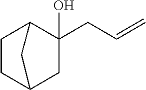 | woody, earthy, metallic, musty, patchouli, wet | 7 | 4 | 1 |
| 4 | 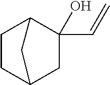 | woody, earthy, gourmand, green, saffron, wet | 5 | 4 | 0 |
| 5 | 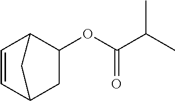 | fruity, herbal, minty, tropical | 7 | 3 | 0 |
| 6 | 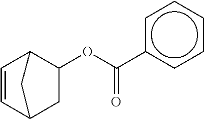 | minty, anisic, lactonic | 6 | 5 | 4 |
| 7 | 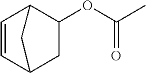 | fruity, tropical, melon, green | 8 | 0 | |
| 8 | 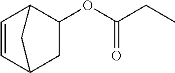 | fruity, tropical, cedarwood, melon, spicy | 7 | 4 | 1 |
| 9 | 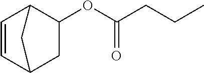 | fruity, tropical, floral, herbal | 6 | 5 | 3 |
| 10 | 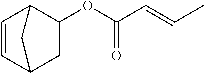 | soulful, gourmand, starchy, cedarwood | 4 | 2 | 2 |
| 11 | 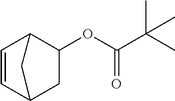 | fruity, cedarwood, berries, tropical, creamy | 7 | 5 | 1 |
| 12 | 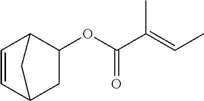 | fruity, tropical, white floral, grassy, melony, wet | 5 | 2 | 1 |
| 13 | 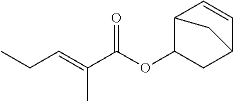 | fruity, green, buttery, pear, plum, floral | 5 | 4 | 1 |
| 14 | 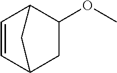 | fruity, tropical, herbal, solventy, melony | 7.5 | 5 | 4 |
| 15 | 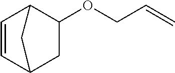 | herbal, woody, apple, cassis, earthy, inky | 7.5 | 6 | 4 |
| 16 | 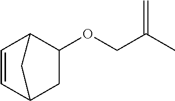 | fruity, tropical, woody, earthy, melony | 7 | 4 | 1 |
| 17 | 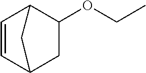 | herbal, minty, green | 8 | 6 | 4 |
| 18 | 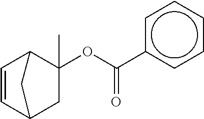 | woody, herbal, leathery, green | 7 | 6 | 4 |
| 19 | 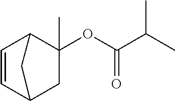 | herbal, minty, fruity, tropical | 5 | 3 | 1 |
| 20 | 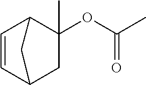 | herbal, minty, floral, lavender | 7 | 4 | 2 |
| 21 | 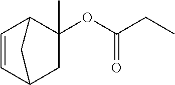 | fruity, lavender, herbal, minty, woody | 5 | 3 | 1 |
| 22 | 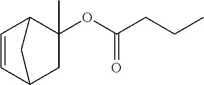 | fruity, minty, herbal, peach, woody | 4 | 3 | 1 |
| 23 | 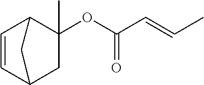 | fruity, mineral, geranium, minty | 7 | 5 | 1 |
| 24 | 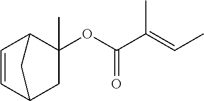 | fruity, cedarwood, herbal | 5.5 | 4 | 2 |
| 25 | 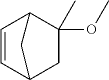 | herbal, green stemmy, marine | 5 | 3 | 1 |
| 26 | 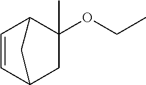 | industrial, metallic, white floral, apple, green | 5 | 2 | 0 |
| 27 | 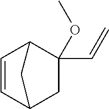 | fruity, green stemmy, woody | 7 | 6 | 5 |
| 28 | 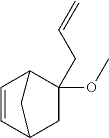 | herbal, green, spicy, woody | 6 | 3 | 0 |
| 29 | 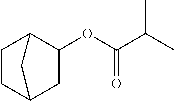 | apple, pear, cherry, almond | 7 | 2 | 1 |
| 30 | 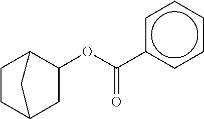 | fruity, woody, green, minty | 5 | 4 | 2 |
| 31 | 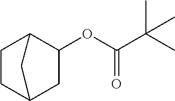 | fruity, rosy, wet | 6 | 2 | 1 |
| 32 | 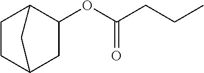 | fruity, woody, white floral, soulful | 6 | 1 | 0 |
| 33 | 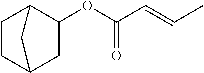 | fruity, apple, pear, soulful | 6 | 2 | 0 |
| 34 | 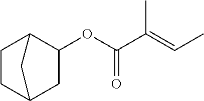 | herbal, solventy, wet, citrus | 6 | 4 | 0 |
| 35 | 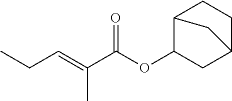 | fruity, apple, pear, green, hay, tobacco | 4 | 5 | 2 |
| 36 | 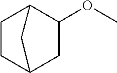 | herbal, lavender, woody, fruity | 7 | 2 | 0 |
| 37 | 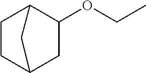 | industrial, mechanical, woody, green, herbal | 7 | 4 | 1 |
| 38 | 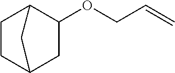 | fruity, solventy, gourmand, basil, green | 6.5 | 4 | 1 |
| 39 | 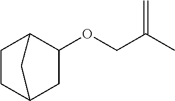 | fruity, driftwood, grassy | 6 | 4 | 1 |
| 40 | 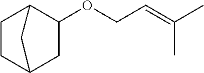 | green, apple, pear, florl, wet, hay/tobacco | 6 | 4 | 1 |
| 41 | 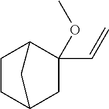 | herbal, fruity, lavender, camphoraceous, piney | 7 | 5 | 1 |
| 42 | 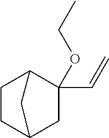 | fruity, ddriftwood, laverder, herbal | 7 | 3 | 1 |
| 43 | 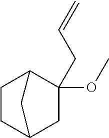 | herbal, green stemmy, marine | 7 | 3 | 1 |
| 44 | 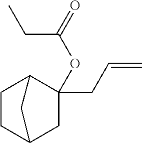 | fruity, herbal, cedarwood, hay/tobacco, liquors | 6.5 | 3 | 1 |
| 45 | 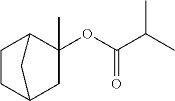 | fruity, apple, pear, spicy, solventy | 6.5 | 4 | 2 |
| 46 | 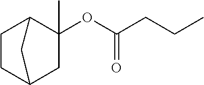 | cedarwoody, lactonic, creamy, milky, tropical | 5 | 4 | 1 |
| 47 | 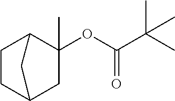 | fruity, mineral, tropical, hay/ tobacco, melony, metallic | 5 | 3 | 0 |
| 48 | 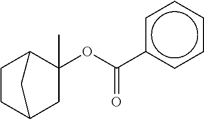 | fruity, tropical, wet, melony, dusty, soulful | 5 | 4 | 2 |
| 49 | 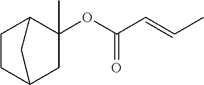 | fruity, herbal, tropical, camphoraceous | 6 | 4 | 1 |
| 50 | 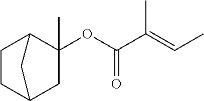 | cedarwood, herbal, peppery, floral, spicy, dried fruit | 5 | 5 | 3 |
| 51 | 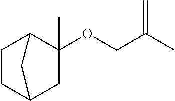 | mineral, cedarwood, driftwood, sandalwood, wet, muguet | 6 | 4 | 1 |
| 52 | 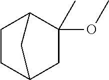 | green stemmy, apple, pear, hay/tobacco | 5 | 5 | 0 |
| 53 | 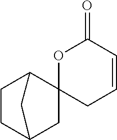 [SMILES: O=C1C=CCC2(CC3CCC2C3)O1] | green, woody, metallic, white floral | 5 | 4 | 3.5 |
| 54 | 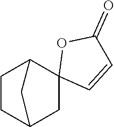 [SMILES: O=C1C=CC2(CC3CCC2C3)O1] | coconut, coumarinic, lactonic, sweet, hay | 6 | 4 | 2 |
| 55 | 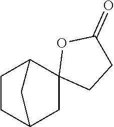 [SMILES: O=C1CCC2(CC3CCC2C3)O1] | coconut, lactonic, coumarinic, sweet, creamy | 5 | 4 | 1 |
| 56 | 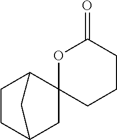 [SMILES: O=C1CCCC2(CC3CCC2C3)O1] | coumarinic, herbal, hay/tobacco | 3.5 | 3 | 3 |
| 57 | 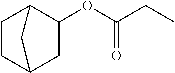 | TBD | TBD | TBD | TBD |

+ = 0-3;
++ = 4-7;
+++ = 8-10

The Examples provided herein are exemplary only and are not intended to be limiting in any way to the various aspects and embodiments of the invention described herein.

What is claimed:

1. A compound of Formula I or Formula II: 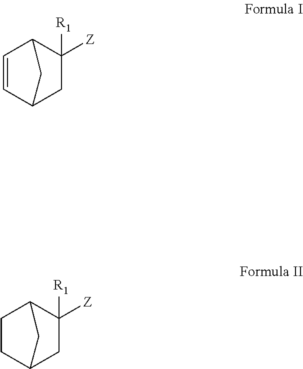
wherein:
R¹ is H or C₁₋₆alkyl, C₂₋₆alkenyl, or C₃₋₆cycloalkyl; or R¹ and Z together form a lactone ring, wherein said lactone ring is optionally substituted by C₁₋₃alkyl and optionally unsaturated;
Z is selected from OR, O—C₂₋₆alkylene-OR, OC(═O)R, O—C₁₋₆alkylene-C(═O)R, O—C₁₋₆alkylene-C(═O)OR; and
R is H, aryl, C₁₋₆alkyl, C₂₋₆alkenyl, or C₃₋₆cycloalkyl;
optionally provided that the compound is not bicyclo[2.2.1]hept-5-en-2-ol.

2. A flavor composition and/or fragrance composition comprising a compound of Formula I or Formula II: 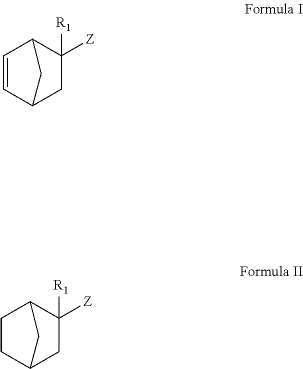
wherein:
R¹ is H or C₁₋₆alkyl, C₂₋₆alkenyl, or C₃₋₆cycloalkyl; or R¹ and Z together form a lactone ring, wherein said lactone ring is optionally substituted by C₁₋₃alkyl;
Z is selected from OR, O—C₂₋₆alkylene-OR, OC(═O)R, O—C₁₋₆alkylene-C(═O)R, O—C₁₋₆alkylene-C(═O)OR; and
R is H, aryl, C₁₋₆alkyl, C₂₋₆alkenyl, or C₃₋₆cycloalkyl;
optionally provided that the compound is not bicyclo[2.2.1]hept-5-en-2-ol, in admixture with one or more non-toxic, orally acceptable, pharmaceutically acceptable, cosmetically acceptable, or acceptable for a household product, carriers or excipients.

3. The composition of claim 2, wherein the compound of Formula I is bicyclo[2.2.1]hept-5-en-2-ol.

4. The composition of claim 2, wherein the compound of Formula I is selected from any of the following: 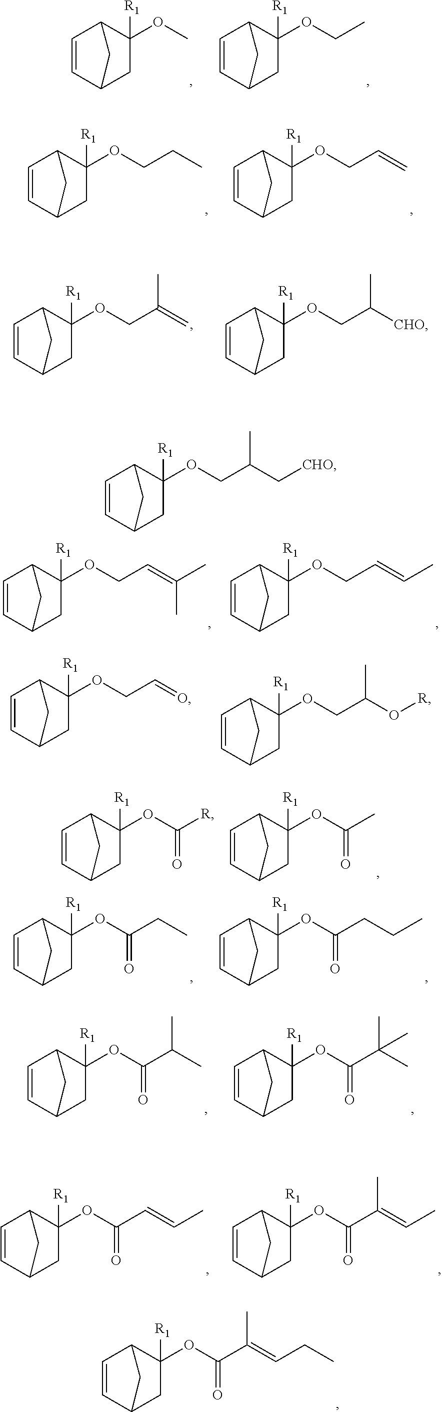 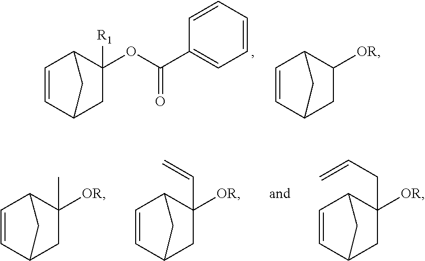
wherein R¹ and R, are as defined in claim 2 (e.g., wherein R¹ and R, are each independently H, methyl, or ethyl).

5. The composition of claim 2, wherein the compound of Formula II is selected from any of the following: 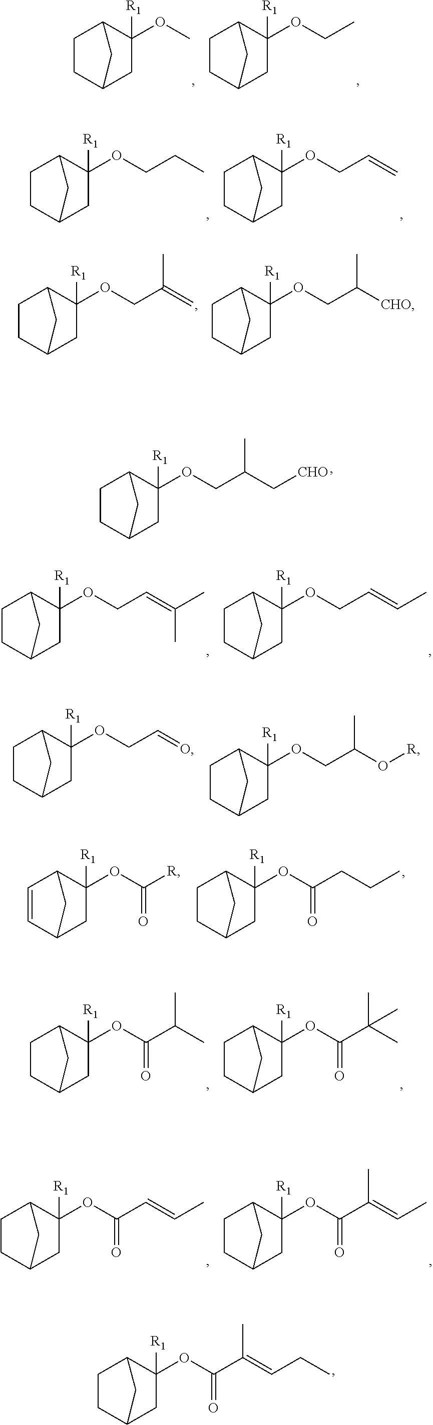 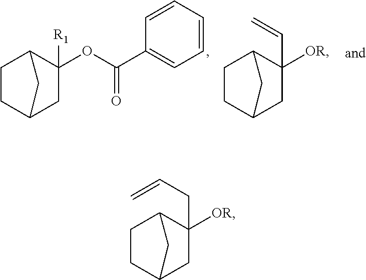
wherein R¹ and R, are as defined in claim 2 (e.g., wherein R¹ and R, are each independently H, methyl, or ethyl).

6. The composition of claim 2, wherein the compound of Formula I is selected from any of the following:

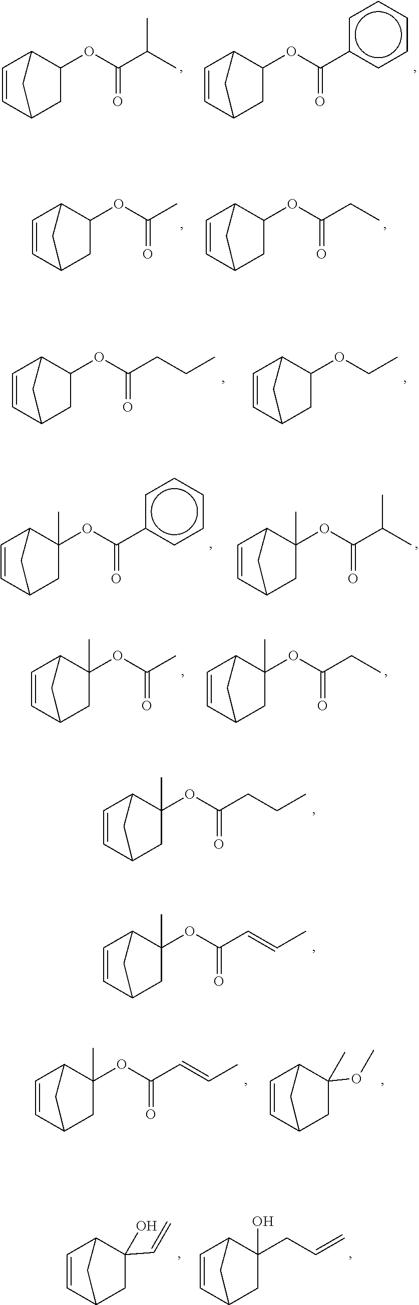

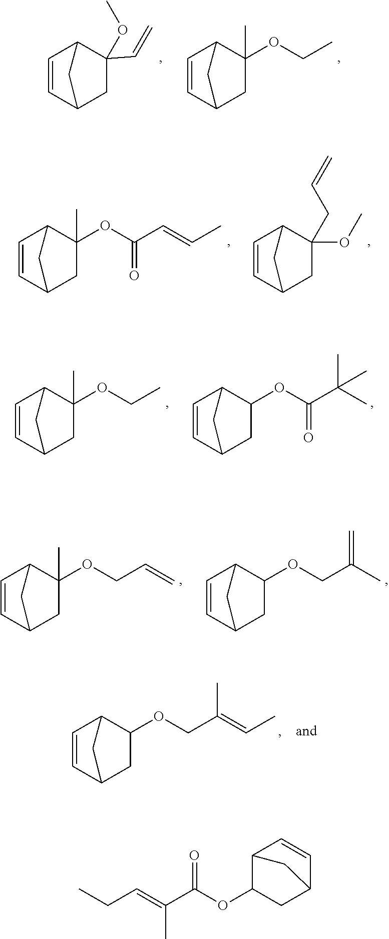

7. The composition of claim 2, wherein the compound of Formula II is selected from any of the following:

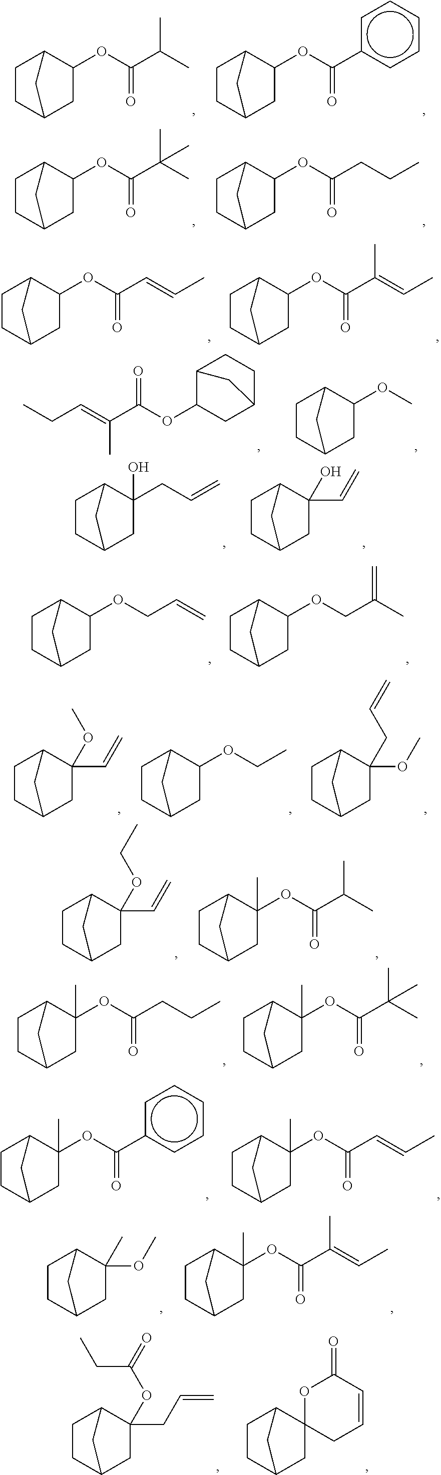

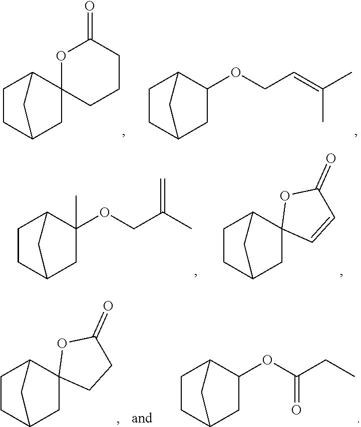

8. The composition of claim 2, wherein the compound of Formula I or II is selected from any of the following:

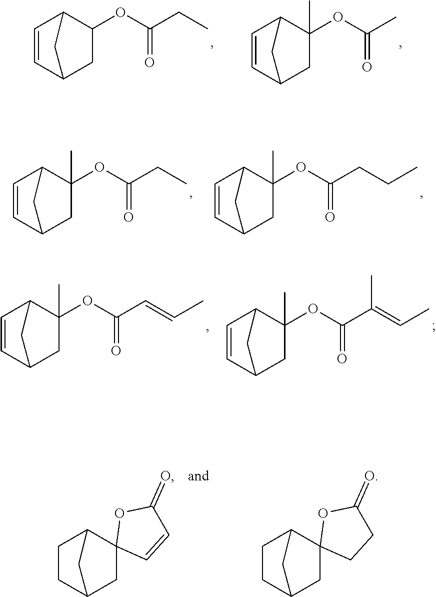

9. The composition of claim 2, wherein the composition further comprises one or more solvents.

10. The composition of claim 2, wherein the composition further comprises one or more other flavors or fragrances.

11. A product comprising the composition of claim 2, e.g., a product selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

12. A compound of Formula I or Formula II selected from any one of the following: [chemical structure]
